(19) United States
(12) Patent Application Publication
(10) Pub. No.: US 2024/0215837 A1
(43) Pub. Date: Jul. 4, 2024

(54) SYSTEM FOR CHARACTERIZATION, DIAGNOSIS, AND TREATMENT OF A HEALTH CONDITION OF A PATIENT AND METHODS OF USING SAME

(71) Applicant: AUTEM MEDICAL, LLC, BEDFORD, NH (US)

(72) Inventors: Frederico Perego Costa, São Paulo (BR); Flavio Soares Correa de Silva, São Paulo (BR); Antonio Francisco Iemma, Piracicaba (BR)

(21) Appl. No.: 18/364,949

(22) Filed: Aug. 3, 2023

Related U.S. Application Data
(63) Continuation-in-part of application No. 15/844,214, filed on Dec. 15, 2017, now abandoned.
(60) Provisional application No. 62/434,779, filed on Dec. 15, 2016.
(60) Provisional application No. 62/572,113, filed on Oct. 13, 2017.

(51) Int. Cl. A61B 5/021; A61B 5/0245

(52) U.S. Cl. CPC A61B 5/02108; A61B 5/02141; A61B 5/0245; A61B 2562/0223; A61B 2562/16

(57) ABSTRACT

A system integrating a hemodynamic parameter (Hdp) monitoring system and a radiofrequency generator synchronized by a processing system is disclosed. The system is capable of identifying health condition-specific Hdp variation value changes in a patient upon the exposure of low-energy amplitude modulated electromagnetic fields frequencies (SFq). The exposure of SFq influences cellular functions or malfunctions in a warm-blooded mammalian subject. The construction of a library of SFq can be used to efficiently and effectively diagnose and treat a health condition in a patient. Methods of using the system are further disclosed.

### CROSS-REFERENCE TO RELATED APPLICATIONS

This application is a continuation-in-part of U.S. patent application Ser. No. 15/844,214, titled SYSTEM FOR CHARACTERIZATION, DIAGNOSIS, AND TREATMENT OF A HEALTH CONDITION OF A PATIENT AND METHODS OF USING SAME, filed Dec. 15, 2017, which claims priority to U.S. Provisional Patent Application No. 62/434,779, SYSTEM FOR CHARACTERIZATION, DIAGNOSIS, AND TREATMENT OF A HEALTH CONDITION OF A PATIENT AND METHODS OF USING SAME, filed Dec. 15, 2016, and U.S. Provisional Patent Application No. 62/572,113 titled SYSTEM FOR CHARACTERIZATION, DIAGNOSIS, AND TREATMENT OF A HEALTH CONDITION OF A PATIENT AND METHODS OF USING SAME, filed Oct. 13, 2017, each of which is incorporated by reference herein in its entirety.

### TECHNICAL FIELD

This invention relates to an electronic system for characterization, diagnosis, treatment, and frequency discovery in a warm-blooded mammalian subject, more particularly involving an integrated system capable of measuring, monitoring, and recording various specified hemodynamic parameter (Hdp) values and computing those values for the identification of specific frequencies thereof for the construction of an intelligent library of frequencies that are capable of causing a programmable frequency generator to expose warm-blooded mammalian subjects to one or more such frequencies in order to diagnose and treat health conditions. The measured and recorded specified Hdp values include values required for purposes of performing the identification of specific frequencies used for the diagnoses and treatment in terms of the invention.

### BACKGROUND

Hdp monitors, or any other similar device capable of registering hemodynamic or cardiac electrical activities, are used to sense and monitor various Hdp values. Such Hdp values can be used to diagnose cardiovascular conditions of a patient. Hdp measurements performed, generally in conjunction with an electrocardiogram (ECG), can include measurements of stroke volume (SV), stroke index (SI), and cardiac output (CO). Such measurements are indicated for the diagnosis and therapy of patients suffering from cardiac conditions, such as heart failure, hypertension, coronary artery disease, and pericardial disease, as well as obstructive lung and pleural disease and renal insufficiency.

Impedance cardiography (ICG), which involves applying a fixed current (e.g., about 400 μA at 40 kHz) to spaced apart electrodes, has been used to measure actual patient current. ICG is essentially concerned with securing CO measurements and comparing the measurements from the ICG procedure with well-known and regularly employed thermodilution (TD) procedures to measure CO or calculate CO by multiplying stroke volume (SV) by heart-rate (HR). In other words, ICG involves the passage of current to a patient solely to attempt to secure, as well as is possible, measurements of CO values for a patient.

Electrocardiogramand photoplethysmography (PPG) involve measuring heart rate metrics. ECG measures the bio-potential generated by electrical signals that control the expansion and contraction of heart chambers; PPG uses a light-based technology to sense the rate of blood flow as controlled by the heart's pumping action.

Heart rate variability (HRV) is the physiological phenomenon of variation in the time interval between heartbeats. It is measured by the variation in the beat-to-beat interval. In order to describe oscillation in consecutive cardiac cycles, other terms have also been used such as heart period variability, RR variability, and RR interval tachogram.

The following steps were used for recording and processing Hdp: Hdp recording, computer digitizing, artifact identification, Hdp data editing, Hdp interval rejection, Hdp data sequence and interpolation, and sampling for time domain heart rate variability and frequency domain heart rate variability.

A summary of the above techniques may be found in Task Force of the European Society of Cardiology and the North American Society of Pacing and Electrophysiology, European Heart Journal 17 (1996) 354-381.

Direct digital synthesis (DDS) is a technique that can be used to ensure stable current sources that can be, but is not necessarily, employed in a component of a hemodynamic monitoring system, such as is described further hereinbelow.

Statistical analyses of differences between CO measurements provided by different measurement procedures can be analyzed by means of Bland-Altman plots. A Bland-Altman plot (difference plot) is a method of data plotting used to analyze the agreement between two different assays, popularized in medical statistics by J. Martin Bland and Douglas G. Altman.

Non-linear methods for the analysis of biometrical signals and Hdp provided by Fourier transformation and the application of Poincaré plots caused by a fluctuating balance between sympathetic and parasympathetic tone at the sino-atrial node. Statistical (time domain) power spectral (frequency domain) and non-linear geometrical analysis for assessing the regulation of the autonomic system.

A summary of the above techniques may be found in J. Fortin et al., Computers in Biology and Medicine 36 (2006) 1185-1203.

Some conventional systems employing one or more of the above techniques can enable a user to diagnose the presence or absence of tumor cell growths or cancers in a patient. In some cases, the identity of the cell growth or tumor can also be identified. However, such systems lack the capability to employ measured Hdp values and HRV to diagnose either a type of cancer, or any other form of a health condition, of a patient.

### SUMMARY

In an embodiment, a system for diagnosing a health condition of a patient may include a hemodynamic parameter (Hdp) monitoring system, an electrically powered frequency generator, and a processing system. The Hdp monitoring system is configured to detect, measure, and record a plurality of first values for each of a plurality of hemodynamic parameters exhibited by a patient during a non-exposure period and a plurality of second values for each of the plurality of hemodynamic parameters exhibited by the patient during or after an exposure period. The exposure period comprises a time period in which the patient is exposed to one or more electromagnetic signals. The electrically powered frequency generator is adapted to generate the one or more electromagnetic signals during the exposure period. The processing system is configured to synchronize the Hdp monitoring system and the frequency generator.

In an embodiment, a system for establishing hemodynamic parameter marker values or hemodynamic parameter surrogate marker values for comparison with patient hemodynamic parameter values stored during treatment of a patient includes a hemodynamic parameter (Hdp) monitoring system and an electrically powered frequency generator. The Hdp monitoring system is configured to detect, measure, and store a plurality of first values for a plurality of hemodynamic parameters exhibited by one or more surrogate patients during a basal or non-exposure period and a plurality of second values for the plurality of hemodynamic parameters exhibited by the one or more surrogate patients during or after an exposure period in which the one or more surrogate patients are exposed to low-energy electromagnetic output signals. The electrically powered frequency generator is adapted to be actuated to generate the low-energy electromagnetic carrier output signals for exposing or applying the low-energy electromagnetic carrier output signals to the surrogate patients during the exposure period.

In an embodiment, a method of diagnosing a health condition of a patient includes measuring, by a hemodynamic parameter (Hdp) monitoring system, a plurality of first values for a plurality of hemodynamic parameters exhibited by a patient during exposure of the patient to highly specific frequency radio frequency (RF) carrier signals and measuring, by the Hdp monitoring system, a plurality of second values for the plurality of hemodynamic parameters exhibited by one or more surrogate patients during exposure of each surrogate patient to the highly specific frequency RF carrier signals. At least one of the one or more surrogate patients may be pre-diagnosed to be healthy or in an identified poor health condition. The method further includes storing the plurality of first values and the plurality of second values, exposing the patient and the one or more surrogate patients to Hdp value-influencing electromagnetic output signals, recording each of the measured Hdp values for each of the hemodynamic parameters measured one or more of before, during, and after exposure of the patient and the one or more surrogate patients to the electromagnetic output signals, processing and analyzing the recorded measured Hdp values to obtain representative Hdp values for each of the recorded Hdp values from the patient and from the one or more surrogate patients, selecting one or more frequencies (SFq) causing significant Hdp value changes in the patient or representative Hdp variation values, storing the SFq and the representative Hdp variation values from a pre-diagnosed or diagnosed patient, and comparing one or more of the SFq and the representative Hdp variation values of the patient with values of the at least one pre-diagnosed surrogate patient, whereby one or more of the SFq, the representative Hdp variation values, the recorded Hdp values of the patient matching a predetermined series of SFq, and the representative Hdp variation values of the pre-diagnosed surrogate patients provide a diagnosis of a health condition of the patient.

In an embodiment, a programmable generator structured to influence cellular functions or malfunctions in a warm-blooded mammalian subject includes a controllable low energy electromagnetic energy generator circuit, at least one data processor, and a connection position. The controllable low energy electromagnetic energy generator circuit is adapted to generate one or more highly specific radio frequency (RF) carrier signals. The generator circuit includes an amplitude modulation (AM) control signal generator adapted to control amplitude modulated variations of the one or more highly specific RF carrier signals and a programmable AM frequency control signal generator adapted to control frequencies at which amplitude modulations are generated. The programmable AM frequency control generator is adapted to control the frequencies to within an accuracy of at least 1000 parts per million relative to a reference AM frequency selected from a range of 0.01 Hz to 150 kHz. The at least one data processor is constructed and arranged to communicate with the at least one generator circuit and to receive control information from a control information source. The connection position is configured to connect to an electrically conductive applicator configured to apply one or more amplitude-modulated low-energy emissions at a program-controlled frequency to the warm-blooded mammalian subject. The reference AM frequencies are selected based on a health condition of the warm-blooded mammalian subject.

### BRIEF DESCRIPTION OF THE DRAWINGS

The accompanying drawings, which are incorporated in and form a part of the specification, illustrate the embodiments of the present disclosure and, together with the written description, serve to explain the principles, characteristics, and features of the disclosure. In the drawings:

FIG. 1 depicts an illustrative schematic structure for an integrated medical system according to an embodiment.

FIG. 2 depicts an illustrative block diagram of an integrated system according to an embodiment.

FIG. 3 depicts an illustrative patient during experimental setup for continuous monitoring of hemodynamic parameters before and during amplitude modulation (AM) radio frequency (RF) electromagnetic field (EMF) exposure according to an embodiment.

FIG. 4 is a table of eleven illustrative hemodynamic parameters simultaneously measured during each heartbeat according to an embodiment.

FIG. 5 is an illustrative flow diagram representing a hemodynamic recording performed continuously during non-exposure and exposure periods in a double-blind fashion according to an embodiment.

FIG. 6A is a series of graphs of Hdp variation values from four different patients diagnosed with hepatocellular carcinoma.

FIG. 6B is a series of graphs of physiological responses in response to reactive and non-reactive pulses for four patients with a diagnosis of hepatocellular carcinoma.

FIG. 6C is a series of graphs demonstrating that different health condition-specific SFq can be defined by linear relationships.

FIG. 6D shows the distribution of disease-specific SFq and healthy-specific SFq during the exposure to three different groups of cancer-specific frequencies in 21 patients.

### DETAILED DESCRIPTION

This disclosure is not limited to the particular systems, devices, and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

As described herein, patient diagnosis may be performed with the aid of measured Hdp values and recorded Hdp values. The recorded Hdp values may be measured in a number of patients that either are pre-diagnosed to be suffering from an identified poor health condition or are in a healthy condition. The Hdp values may be stored at determined times and for determined periods of time, as described in greater detail below.

In an embodiment, a system includes an Hdp monitoring system used to measure and record Hdp values and a frequency generator configured to provide one or more highly specific frequencies to a patient via radio frequency (RF) carrier signals.

In an embodiment, the system may identify specific electromagnetic field amplitude modulated frequencies (SFq), which are a subgroup of the highly specific frequency RF carrier signals. The SFq may be used to influence cellular functions or malfunctions in a warm-blooded mammalian subject. The exposure of a warm-blooded mammalian subject to the SFq may cause representative Hdp variation values to change in a manner that indicates whether or not one or more highly specific frequency RF carrier signals have a potential biological effect in the warm-blooded mammalian subject. The specificity of changes in representative Hdp variation values may be a surrogate marker for the diagnosis and treatment of the warm-blooded mammalian subject.

In an embodiment, the system may store one or more groups of identified SFq in a server connected by a protected Internet-based platform to form an intelligent library of SFq (ILf). The stored data may be combined, organized, compared, and characterized for use in the diagnosis of patients or individuals and in the treatment of health conditions in patients having similar diagnoses.

The integrated frequency generator used to emit or expose a warm-blooded mammalian subject to one or more highly specific frequency RF carrier signals may be a programmable generator and may be an electronic component that is activatable by electrical power as part of an integrated system. The programmable generator may be employed to influence cellular functions or malfunctions in a warm-blooded mammalian subject. The programmable generator may include one or more controllable low energy electromagnetic energy generator circuits configured to generate one or more highly specific frequency RF carrier signals. One or more microprocessors or integrated circuits that include or communicate with the one or more generator circuits are provided. In an embodiment, the one or more microprocessors may also be used to control the transmission and reception of control information from a processing system. In an embodiment, the one or more generator circuits may include one or more amplitude modulation (AM) frequency control signal generators configured to control amplitude modulated variations of the one or more highly specific frequency RF carrier signals. The one or more generator circuits may further include one or more programmable AM frequency control signal generators configured to control the frequency at which the amplitude modulations are generated.

The system may further include a processing system configured to integrate and synchronize the Hdp monitoring system and the one or more programmable generators. Various Hdp values measured and recorded by the Hdp monitoring system while exposing a warm-blooded mammalian subject to one or more highly specific frequency RF carrier signals emitted by the one or more programmable generators may be processed by the processing system. The information resulting from such processing may be stored, for example, in the ILf. The processing system may further control, synchronize, and load a control program into the one or more programmable generators with a specific series of SFq. As such, the processing system may integrate and synchronize the Hdp monitor, the ILf, and the one or more programmable generators to support an integrated solution.

In an embodiment, the processing system and the ILf may be part of a server connected to the remainder of the system by a protected web platform. The ILf may include an artificial intelligence capability used for storing, combining, organizing, comparing, characterizing, and processing SFq and recorded representative Hdp variation values. The ILf may store and organize a series of SFq and representative Hdp variation values identified in a warm-blooded mammalian subject or patient. One or more series of SFq may then be loaded into the one or more programmable generators. The one or more programmable generators may accurately control the emission of the frequency of the amplitude modulations with an accuracy of at least 1000 parts per million (ppm) relative to one or more determined or predetermined reference AM frequencies. In an embodiment, the AM frequencies may be within a range of 0.01 Hz to 150 kHz. The processing system may further include a connection or a coupling position. The connection or coupling position may be used to connect or couple the processing system to an electrically conductive applicator that applies the one or more amplitude-modulated low-energy emissions at the accurately controlled modulation frequencies to the warm-blooded mammalian subject.

Through the course of performing numerous clinical trials in which multiple measurements of various Hdp values in patients have been recorded, it has been determined that such Hdp values differ based on the type of health condition that a patient faces. In particular, different Hdp values have been identified with respect to different types of cancer. Such determinations have provided a basis for proposing a diagnostic procedure based on measured Hdp values that is used to diagnose a particular form of cancer in a patient. These determinations further suggested that many health conditions suffered by a patient, including viral, parasitic, or other pathogenic invasions or organ dysfunctions, which could lead to toxins being present in the blood of a patient. Drug abuse, poisons, high low-density lipoprotein (LDL) cholesterol levels, venom from a snake-bite, and the like, may be diagnosed in a patient on the basis of certain identified measured Hdp values.

A frequency synthesizer may be used to generate a particular frequency or a series of frequencies with precision. For example, a user may use a keyboard or other input device to select one or more frequencies, which in turn may cause a circuit to turn a generated signal ON or OFF within well-defined time intervals.

In an embodiment, a processing system processes Hdp values measured and recorded by an Hdp monitoring system connected to a warm-blooded mammalian subject during the exposure to one or more highly specific frequency RF carrier signals emitted by a programmable generator. The Hdp monitoring system may measure and record Hdp values for further processing. The processing system may incorporate one or more algorithms that analyze the recorded Hdp values obtained by the Hdp monitoring system. The processing system uses various measured and recorded Hdp values of the subject and identifies SFq, characterized by recognizable patterns of Hdp variation value changes, herein referred to as representative Hdp variation values. The SFq is a subset of the highly specific frequency RF carrier signals that influence cellular functions or malfunctions in a warm-blooded mammalian subject (i.e., a patient). The processing system generally identifies the various measured and recorded Hdp values of the patient using electrodes placed in topical contact with various determined parts of the body as part of the Hdp monitoring system. The Hdp monitoring system further comprises a recording component that records the various identified measured Hdp values of the patient. In an embodiment, the recording component may store the measured Hdp values in a storage device of the Hdp monitoring system. In an alternate embodiment, the recording component may store the measured Hdp values in any storage device on which the various identified measured Hdp values of the patient can be recorded for immediate and/or future processing. Hdp values may include the values of, for example, one or more of the following hemodynamic parameters:
RR interval (interval from an R peak to the next R peak as shown, for example, on an electrocardiogram (ECG)) (RRI);
heart rate (HR);
systolic blood pressure (sBP);
diastolic blood pressure (dBP);
median blood pressure (mBP);
pulse pressure (PP);
stroke volume (SV);
cardiac output (CO); and
total peripheral resistance (TPR).

In an embodiment, the processing system may include a device synchronizer, a data aggregator, a storage device and/or a storage interface, and an interface controller. The interface controller may be responsible for matching the Hdp values and the exposure to one or more highly specific frequency RF carrier signals (synchronization). Additionally or alternatively, the interface controller may be responsible for consolidating the records (data aggregation) to be stored (storage) for further processing (interface controller) in such a way that Hdp values are linked to an exposure to one or more highly specific frequency RF carrier signals from which such Hdp values were measured. The Hdp monitoring system and the programmable generator may be connected via the interface controller. The modules corresponding to synchronization and data aggregation and the storage interface may be packed as a portable integrated hardware solution.

In another embodiment, the processing system may include, inter alia, two components: a statistical mining component and a machine learning/evolutionary game theory component. It should be noted that, as used herein, machine learning refers to various types of machine learning including, for example, deep machine learning and layered machine learning. The statistical mining component may include a series of mathematical procedures based on discriminant analysis and support vector machine (SVM). Hdp values may be constant selected metric variables and their dependent new attributes that are analyzed based on different well-established statistical methods. Using multivariate discriminant analysis and other coordinate transformation based on relevant component analysis, Hdp values may be represented as centroids of representative Hdp variation values with well-defined threshold values in order to optimize common metrics. The machine learning/evolutionary game theory component may include permanently refined, cluster analysis and updated mathematical algorithms that, or by new discriminating attributes, perform cutoff refining to (1) identify patterns of responses for health condition-specific frequencies, herein named representative Hdp variation values and (2) store representative Hdp variation values and the corresponding health condition-specific frequencies. These components may be implemented on a central and secure server-side system connected to the integrated hardware solution via encrypted communication over a network, such as the Internet.

In yet another embodiment, an intelligent library of SFq may be located in the central and secure server system. In an embodiment, the library may be connected to all instances of the integrated hardware solution via encrypted communication over a network, such as the Internet. In such an embodiment, the network solution may provide a real-time, integrated, and evolutionary system combining all working devices. Permanent, updated, de-identified patient's demographic and clinical information data gathered from physician and patient-reported outcomes combined with records of representative Hdp variation values and the correspondent health condition-specific frequencies (SFq) and the data may be stored in the ILf. Threshold values for representative Hdp variation values may be refined based on newly added values. Such data may be structured and processed to refine the procedures for diagnosis, treatment, and follow-up for a health condition of a patient. ILf may have computing capabilities to support statistical data mining and machine learning for pattern recognition and evolutionary game theory for identification of points of equilibrium, which characterize the best possible matching for each series of SFq and/or representative Hdp variation values and correspond to the diagnosis and treatment outcome information. Refinement procedures are implemented as artificial intelligence based meta-programs, which take into account patient segmentation. The programmable generator is connected by the interface controller in the processing system in order to transfer data between the processing system and the programmable generator. Refined procedures are then downloaded back to the processing system module of the integrated hardware solution, in order to re-program the programmable generator.

The interface controller may connect the programmable frequency generator to the processing system in order to allow for the transfer of data. Refined procedures may be downloaded to the processing system in order to update the programmable frequency generator prior to or during a treatment session.

In an embodiment, a diagnosis of a health condition of a patient may be determined based on one or a group of SFq and/or representative Hdp variation values identified by the processing system. In an embodiment, a plurality of measured and recorded Hdp values may be submitted to the processing system during the exposure of the patient to one or more highly specific frequency RF carrier signals. The processing system may identify SFq and/or representative Hdp variation values in a patient diagnosed with a health condition. In an embodiment, the identified SFq and representative Hdp variation values may be stored in the ILf. The warm-blooded mammalian subject, during exposure to a selected group of SFq (i.e., a subgroup of highly specific frequency RF carrier signals emitted by a programmable generator) may have various Hdp values that are measured and recorded by the Hdp monitoring system processed to identify the characteristic hemodynamic response pattern to SFq exposure. The processing system identifies representative Hdp variation values related to the selected group of SFq. The processed information may be stored in the ILf for instant and/or future database comparisons. The diagnosis identification may be the result of searching for patterns of response that are consistent with a specific health condition of a patient. The processing system may diagnose a health condition of a patient by incorporating a series of mathematical algorithms that analyze the recorded Hdp data obtained by the Hdp monitoring system.

In another embodiment, a user may be enabled to search for SFq. The searching procedure may be conducted during the exposure of a patient to one or more highly specific frequency RF carrier signals. For example, searching for SFq may include a process that involves the Hdp monitoring system reviewing measured and recorded Hdp stored in the processing system during exposure of the patient to one or more highly specific frequency RF carrier signals. The searching procedure for SFq may involve the application of mathematical algorithms to determine a series of specific frequencies to be provided by the programmable generators. In an embodiment, the searching process may include processing of measured and recorded Hdp values by the Hdp monitoring system during the exposure to a series of specific frequencies, such as a subgroup of highly specific frequency RF carrier signals, produced by a programmable generator in a warm-blooded mammalian subject with an unknown health condition or a patient with a known health condition. With respect to the exposure of a subject or patient to a predetermined sequence of one or more highly specific frequency RF carrier signals, the term “accurately controlled” means that modulated low-energy electromagnetic emissions are modulated to within a resolution of at most about 1 Hz of higher frequencies (greater than about 1000 Hz). For example, if a determined or predetermined modulation frequency to be applied to the warm-blooded mammalian subject is about 2000 Hz, accurate control of such modulated low-energy emission requires the generated frequency to be between about 1999 Hz and about 2001 Hz. The processing system identifies SFq and representative Hdp variation values during the searching procedure.

In an embodiment, new SFq may be discovered. The discovery procedure may be conducted during exposure of an individual or patient to one or more highly specific frequency RF carrier signals. Discovery of new SFq may include having the processing system receive measured and recorded Hdp values from the Hdp monitoring system during exposure to one or more highly specific frequency RF carrier signals. The discovery of new SFq may further involve the application of mathematical algorithms to determine a series of specific frequencies by the programmable generators. In an embodiment, the searching process may process measured and recorded Hdp values from the Hdp monitoring system during exposure to a series of specific frequencies that are a subgroup of highly specific frequency RF carrier signals produced by a programmable generator in a warm-blooded mammalian subject with a known health condition. In an embodiment, the processing system identifies SFq and representative Hdp variation values during the process of discovering new SFq.

In an embodiment, a diagnosis of a health condition of a patient may be determined based on one or a group of SFq and/or representative Hdp variation values identified by the processing system. In an embodiment, a plurality of measured and recorded Hdp values may be submitted to the processing system during the exposure of the patient to one or more highly specific frequency RF carrier signals. The processing system may identify SFq and/or representative Hdp variation values in a patient diagnosed with a health condition. In an embodiment, the identified SFq and representative Hdp variation values may be stored in the ILf. The warm-blooded mammalian subject, during exposure to a selected group of SFq (i.e., a subgroup of highly specific frequency RF carrier signals emitted by a programmable generator) may have various Hdp values that are measured and recorded by the Hdp monitoring system processed to identify the characteristic hemodynamic response pattern to SFq exposure. The processing system identifies representative Hdp variation values related to the selected group of SFq. The processed information may be stored in the ILf for instant and/or future database comparisons. The diagnosis identification may be the result of searching for patterns of response that are consistent with a specific health condition of a patient. The processing system may diagnose a health condition of a patient by incorporating a series of mathematical algorithms that analyze the recorded Hdp data obtained by the Hdp monitoring system.

In another embodiment, a user may be enabled to search for SFq. The searching procedure may be conducted during the exposure of a patient to one or more highly specific frequency RF carrier signals. For example, searching for SFq may include a process that involves the Hdp monitoring system reviewing measured and recorded Hdp stored in the processing system during exposure of the patient to one or more highly specific frequency RF carrier signals. The searching procedure for SFq may involve the application of mathematical algorithms to determine a series of specific frequencies to be provided by the programmable generators. In an embodiment, the searching process may include processing of measured and recorded Hdp values by the Hdp monitoring system during the exposure to a series of specific frequencies, such as a subgroup of highly specific frequency RF carrier signals, produced by a programmable generator in a warm-blooded mammalian subject with an unknown health condition or a patient with a known health condition. With respect to the exposure of a subject or patient to a predetermined sequence of one or more highly specific frequency RF carrier signals, the term “accurately controlled” means that modulated low-energy electromagnetic emissions are modulated to within a resolution of at most about 1 Hz of higher frequencies (greater than about 1000 Hz). For example, if a determined or predetermined modulation frequency to be applied to the warm-blooded mammalian subject is about 2000 Hz, accurate control of such modulated low-energy emission requires the generated frequency to be between about 1999 Hz and about 2001 Hz. The processing system identifies SFq and representative Hdp variation values during the searching procedure.

In an embodiment, new SFq may be discovered. The discovery procedure may be conducted during exposure of an individual or patient to one or more highly specific frequency RF carrier signals. Discovery of new SFq may include having the processing system received measured and recorded Hdp values from the Hdp monitoring system during exposure to one or more highly specific frequency RF carrier signals. The discovery of new SFq may further involve the application of mathematical algorithms to determine a series of specific frequencies by the programmable generators. In an embodiment, the searching process may process measured and recorded Hdp values from the Hdp monitoring system during exposure to a series of specific frequencies that are a subgroup of highly specific frequency RF carrier signals produced by a programmable generator in a warm-blooded mammalian subject with a known health condition. In an embodiment, the processing system identifies SFq and representative Hdp variation values during the process of discovering new SFq.

In yet another embodiment, the system may be used to construct and update the ILf. The process used to construct and update the ILf library with frequencies may use the processing system to identify SFq and/or representative Hdp variation values in warm-blooded mammalian subjects. The processing system may store identified SFq and representative Hdp variation values in a central server connected by a protected Internet platform. The ILf may store newly identified SFq from warm-blooded mammalian subjects with a known health condition. The stored SFq that are originated from warm-blooded mammalian subjects with known health conditions may undergo artificial intelligence processing to allow future diagnosis, identification, and treatment program generation for the treatment of patients diagnosed with the same health conditions. For example, one or more SFq identified in a patient diagnosed with a specific health condition may be used with other warm-blooded mammalian subjects for diagnosis and treatment proposes.

In another embodiment, a patient may be treated using the present system. The treatment procedure may include exposing patients to one or more highly specific frequency RF carrier signals. The programmable generators may be loaded with a program control in order to produce a selected and health condition-specific group of SFq that are to be provided to the warm-blooded mammalian subjects with the specific health condition. The ILf may store and update a plurality of selected groups of SFq identified in warm-blooded mammalian subjects with the same health conditions. The processing system may load the programmable generators with program controls to expose warm-blooded mammalian subjects with selected health condition to the specific group of SFq. In an embodiment, the group of SFq may be accurately controlled. In an embodiment, the group of SFq may have a resolution of about 0.5 Hz from the intended determined or predetermined modulation frequency. In another embodiment, the group of SFq may have a resolution of about 0.1 Hz from the intended determined or predetermined modulation frequency. In yet another embodiment, the group of SFq may have a resolution of about 0.01 Hz from the intended determined or predetermined modulation frequency. In still another embodiment, the group of SFq may have a resolution of about 0.001 Hz from the intended determined or predetermined modulation frequency.

In an embodiment, the system may be used to provide follow-up treatment to a patient. The follow-up procedure may include testing procedures in patients under treatment for a heath condition during a determined period of time or real-time testing in patients during the exposure to one or more highly specific frequency RF carrier signals while under a treatment cycle. The follow-up testing procedure may be conducted during the exposure to one or more highly specific frequency RF carrier signals in an individual or patient. The follow-up testing of a patient with a known health condition may involve the application of mathematical algorithms to determine a series of specific frequencies to be loaded into the one or more programmable generators. The follow-up testing may include providing the processing system with measured and recorded Hdp from the Hdp monitoring system during exposure to one or more highly specific frequency RF carrier signals for follow-up comparison. The processing system may identify SFq and representative Hdp variation values during the follow-up testing procedure that may or may not modify as result of the treatment to a patient. The follow-up testing procedure result may be able to identify patterns of response consistent with a non-invasive prediction of treatment response of a health condition. The processing system may incorporate a series of mathematical algorithms that analyze the recorded Hdp data obtained by the Hdp monitoring system.

Of importance is the exposure of SFq emissions to be at a very low and safe energy level and result in low levels of absorption, the reason believed to be that physiological exchanges or flow of electrical impulses within warm-blooded animals (which are to be affected by application of the emissions of the present invention) are similarly at very low energy levels. In any event, in the region (at or near to the position of contact or close-by induction of the electrically conductive applicator with a subject receiving treatment), the specific absorption rate (SAR) should be and is most preferably substantially less than 1.6 mW/g weight of living tissue.

Furthermore of importance to achieve the intended biological therapeutic effect is that the stability of the emissions be maintained during emission and that such stability should preferably be of the order of 10⁻⁵, more preferably 10⁻⁶, and most preferably 10⁻⁷, stability being determined as the relative deviation of frequency divided by the desired frequency, e.g., 0.01 Hz (deviation)/1,000 Hz (desired freq.)=10⁻⁵.

The exposure of one or more highly specific frequency RF carrier signals by the programmable generators is integrated into the invention. The programmable generator is an electronic component with significant improvements from a patented medical device that includes a microprocessor (which may, more recently, be replaced by an integrated circuit). The programmable generator in the invention uses control information that is loaded from the processing system. The other improvement in the programmable generator in the invention is the integrated part of the invention, which combines other medical devices such as a Hdp monitoring system and other computing servers, with all of them operating together and synchronized by the processing system as one single new medical device. As a result, the new and improved programmable generator can be instantly loaded with updated series of specific SFq identified in a single warm-blooded mammalian subject or identified in a group of warm-blooded mammalian subjects with same health conditions. In addition, the new and improved programmable generator in the invention supports different applications besides treatment of a patient, such as diagnosis, searching for SFq, and follow-up of a treatment. The microprocessor (or now alternatively integrated circuit) then controls the function of the programmable generator to produce the desired therapeutic emissions. Also described is the provision in the programmable generator of an impedance transformer connected intermediate the emitter of low-energy electromagnetic emissions and a probe (here more broadly described as an electrically conductive applicator) for applying the emissions to the patient. The impedance transformer substantially matches the impedance of the patient seen from the emitter circuit with the impedance of the output of the emitter circuit.

The Hdp monitoring system is a currently available and patented medical device with different brands used in different applications that is integrated in the present invention. The Hdp monitoring system is necessary for measurement and recording of Hdp values used by the processing system for SFq identification. The identified SFq is used for diagnosis and treatment of a health condition of a patient. The system integrates the Hdp monitoring system that measures and records various identified Hdp values of the patient. The system generally measures various identified Hdp values of the patient utilizing electrodes placed in topical contact with various determined parts of the body. The Hdp monitoring system further comprises recording means that records the various identified Hdp values of the patient. The recording means can utilize any storage device on which the various identified measured Hdp values of the patient can be recorded. The storage of measured and recorded Hdp, in accordance with the invention, require that a variety of Hdp values need to be measured and recorded, which include the values of at least the following nine Hdp's:
RR interval (interval from the R peak to the next as shown on an electrocardiogram (ECG) (RRI);
heart rate (HR);
systolic blood pressure (sBP);
diastolic blood pressure (dBP);
median blood pressure (mBP);
pulse pressure (PP);
stroke volume (SV);
cardiac output (CO); and
total peripheral resistance (TPR).

The Hdp values are measured and recorded following established procedures. Initial measurements during non-exposure highly specific frequency RF carrier signals periods in an individual or patient or Hdp values thereof are herein named basal measurements or basal Hdp values. In terms of procedure, initial measurements of above parameters are performed on warm-blooded mammalian subjects after a period of relaxation, for example about 15 minutes, while the patient is lying in a supine position (face and preferably also palms of the hands facing upwardly) or in another comfortable and relaxed position.

After having performed the above initial measurements, the diagnosed or pre-diagnosed warm-blooded mammalian subjects are exposed to or application of the above-described procedures, i.e. diagnosis, searching for SFq, discovering new SFq, treatment's follow-up involving exposure to or application of selected series of one or more highly specific frequency RF carrier output signals, thereof are herein named exposure measurements or exposure Hdp values.

The above-mentioned one or more highly specific frequency RF carrier signals are electromagnetic field frequency (EMF) output signals that may be produced by a loaded control program into the programmable generator capable to generate EMF output signals at certain predetermined amplitude modulation (AM) frequencies. The subjects or patients are most preferably exposed to or the EMF output signals are applied to patients during heart-beat times over a determined period of time, most preferably over the time of at least ten heart-beat times of the patient or a period of at least 10 seconds. This procedure is part of the integrated solution of the invention, and it would in general take place while the patient remains connected to or is reconnected to both the synchronized Hdp monitoring system and programmable generator of the system of the invention so that Hdp values may be measured and recorded during the period of exposure or application. The Hdp values may, however, also or alternatively be the data source to identify SFq and/or representative Hdp variation values after software processing described above.

The above Hdp values measured during or after above exposures or applications to subjects or patients are herein referred to as exposure or exposure Hdp values and post-exposure or post-exposure Hdp values, respectively.

The procedures above, in general, as applied to multiple patients pre-diagnosed or diagnosed patients with known health conditions to be suffering from an identified form of poor health condition, provide multiple basal Hdp values, multiple exposure Hdp values and multiple post-exposure Hdp values as related to the identified pre-diagnosed or diagnosed form of poor health condition. These multiple Hdp values, for example, for most if not all of the nine Hdp parameters listed above, may in general be somewhat scattered values. Accordingly, for purposes of defining representative Hdp values, such scattered values would regularly be submitted to the processing system that integrates multiple mathematical calculations for purposes of identifying SFq and representative Hdp variation values.

In line with above, the Hdp monitoring system is only part of the present invention, providing means for software processing Hdp values for use in the identification of SFq and the diagnosis of health conditions of a patient. The identification of SFq and representative Hdp variation values are termed representative surrogate markers that are determined through the processing system from the Hdp monitoring system that measures and records Hdp values performed during non-exposure and exposure periods on patients pre-diagnosed or diagnosed as either healthy or suffering from a known form of poor health condition.

Representative surrogate markers employed for diagnosis, searching for SFq, treatment, and treatment's follow-up purposes, in terms of the present invention, are derived from computative combinations of information from both representative basal Hdp measured values, representative exposure Hdp measured values, and representative post-exposure Hdp values. Since the exposure EMF frequencies employed for influencing Hdp values are different for each health condition, and post-exposure Hdp values are similarly different, the computative combinations for deriving representative surrogate markers for a specified health condition requires different computations using discriminant analysis and well-established statistical methods for ideal threshold values determination.

The reliability of representative surrogate marker values is of course dependent on the number of pre-diagnosed and diagnosed surrogates included for each type of poor health condition examined. Thus, the incidence of poor health conditions among populations, more particularly high incidence of poor health conditions that are difficult to diagnose, such as Hepatocellular Carcinoma (HCC) or related liver diseases, has received particular attention. Similarly, the relatively high incidence of breast cancer has thus far also received particular attention, as reported below.

Post-exposure Hdp measured values, insofar as may be reflected following on exposure to or application of low-energy EMF carrier signals, in terms of the invention, may be compared with Hdp values that occur following an exposure or application to a patient of predetermined EMF frequency values predetermined to alleviate a cause of a specified poor health condition of a patient. Matching basal, exposure, and post-exposure Hdp values, on their own or after using the processing system, may support the efficacy of treatment by application of said predetermined EMF carries signals and provide a preliminary indication of diagnosis of the health condition of the patient. Reference to the further scientific details related, for example, specifically to two different forms of cancer diagnoses are described below. Here, mention is made to a patient's diagnosis following the basal non-exposure period and correlations of patient's diagnosis with hemodynamic patterns in male HCC and female breast cancer in comparison with healthy controls. Similarly, mention is made to tumor-specific hemodynamic response pattern during exposure periods.

The time periods of exposure or application of EMF frequency output signals by means of a variable frequency programmable generator device within a broad range of frequencies, for example, EMF frequencies within a range between from about 0.01 to about 150 MHz, may require a short period of time for Hdp values to be varied at any particular frequency value. Thus, consecutive exposures or applications of sections of the range of EMF frequencies may be required in order to identify EMF frequency values at which basal, exposure, and post-exposure Hdp value variations actually occur during the heart-beat times at which Hdp values are measured and recorded by the Hdp monitoring system and processed by the processing system.

The system of the invention includes, besides programmable generator of EMF frequency output signal, the processing system and the ILf central server, the output signal frequency measurement and recording means for measuring and recording such frequency values at which frequencies Hdp variances of at least certain of the Hdp values are exhibited, herein threshold values. Similarly, Hdp value recording means for recording each of the measured values for each of the identified Hdp's, preferably separately of one another, measured and recorded before, during, or after the period of time of exposure to or application of output signals to the patient.

A further component of the integrated invention, additional to those described above, is the processing system component that may be integrated with or coupled to the recording means for recording Hdp values before, during, or after performing or exposing the patient to a cellular excitation procedure. The processing system component may include program-controlled calculation means for performing a series of mathematical analysis of various of the recorded Hdp values to obtain representative surrogate values, such as the identification of SFq and representative Hdp variation values, for each of the different recorded Hdp values, optionally making a determination of ratios between different representative Hdp values and comparing either or both of such representative values or ratios between different values with predetermined representative values or ratios (threshold values) characteristic of an SFq and/or representative Hdp variation value changes while exposing the patient to a cellular excitation procedure, predetermined in patients known to be either healthy or suffering from, or likely to develop, an identified poor health condition. The comparison of calculated representative surrogate values, such as recorded Hdp values or ratios and identified SFq and/or representative Hdp variation values in patients diagnosed with the same health condition, which match with predetermined representative Hdp values or ratios and/or identified SFq and/or representative Hdp variation values, leads to providing an indication of a diagnosis of a health condition of a patient.

The processing system component may, alternative or additional to being integrated or coupled to the recording means as described above, be located at a central server connected to the invention by a protected web platform, which may perform the analysis based on recorded Hdp information received or communicated to the center.

The exposure of identified SFq has demonstrated biological activity and supports its use as a novel treatment modality (Examples 6 and 7).

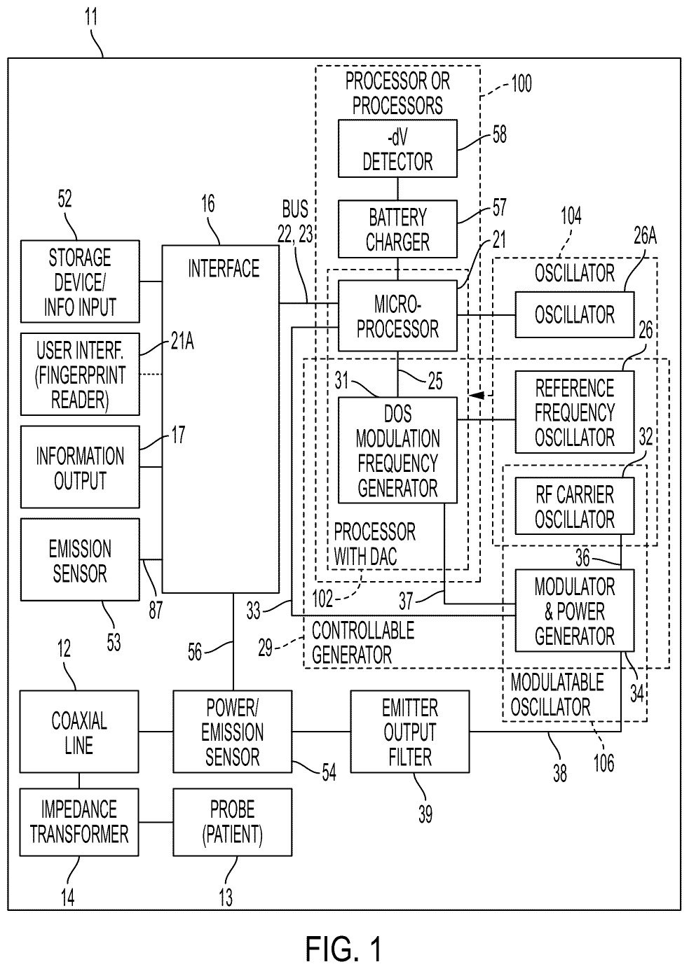

FIG. 1 depicts an illustrative schematic structure for an integrated medical system according to an embodiment. As shown in FIG. 1, a system for diagnosing a health condition of a patient integrates an Hdp monitoring system and a frequency generator. In an embodiment, the system may further include a processing system.

The Hdp monitoring system determines a cardiovascular performance reserve for each individual patient. In an embodiment, the Hdp monitoring system may receive input physiological data from a patient. The input physiological data may be used to obtain a parameter Z which is or approximates a product of the patient's Stroke Volume (SV) and the patient's Systemic Vascular Resistance (SVR). The Hdp monitoring system may further provide a value representing the Respiratory Rate (RR) of the patient. The RR value may be determined by one or more of a measurement using a dedicated device, a calculation performed using the input physiological data or manually by using best estimate, such as making an estimate based on the heart rate of the patient.

A modulated low-energy electromagnetic emission application system generator may be used to emit low energy radio frequency (RF) electromagnetic waves to a warm-blooded mammalian subject. The low energy RF electromagnetic waves may be used to treat a warm-blooded mammalian subject suffering some limited number of described health conditions. The system described herein integrates and synchronizes the Hdp monitoring system and the generator via the processing system, which may further be connected to a central server by a protected web-based platform.

The system described herein may be an integrated solution having a patient side component and a server-side component. The patient side component may include the Hdp monitoring system connected to the programmable generator. Both the Hdp monitoring system and the programmable generator may be connected to the processing system to enable synchronization of the devices and allow for compatible data aggregation. The central server-side component may be connected with the patient side component via the protected web based platform and may provide artificial intelligence based computation and data storage.

The two components of the system may enable bidirectional data transfer in real-time as described above. For example, once the programmable generator is loaded with one or more control programs of selected series of SFq, the programmable generator may be disconnected from the integrated solution to enable outpatient use. The programmable generator may be reconnected in the integrated solution to permit batch upload of update data and to allow automatic treatment profiling to be transferred back to the processing system.

Referring to FIG. 1, the new medical device related to the invention is the integration of other medical devices. The Hdp monitor provides a method for determining a cardiovascular performance reserve for each individual patient, comprising the steps of a) receiving input physiological data from the patient for obtaining a parameter Z, which is or approximates the product of the Stroke Volume (SV) by the Systemic Vascular Resistance (SVR); b) providing a value representing the Respiratory Rate (RR) of said patient, wherein the Respiratory Rate (RR) value is provided by measurements using dedicated device(s), calculations from the input physiological data, or manually by using best estimate, as described in U.S. Patent Application Publication No. 2015/0005647 c) Electrocardiogramand photoplethysmography (PPG), where ECG measures the bio-potential generated by electrical activity of the heart and PPG senses the rate of blood flow, d) Heart rate variability (HRV) that is oscillation in consecutive cardiac cycles.

Electromagnetic emission application system 11 generator relates to the practice of emissions of low energy radio frequency (RF) electromagnetic waves to a warm-blooded mammalian subject for treating a warm-blooded mammalian subject suffering some limited number of described health conditions as described in prior U.S. Pat. Nos. 4,649,935 and 4,765,322. The new device related to the invention integrates and synchronizes both medical devices by the processing system that connects the new device to a central server by protected web-based platform.

Referring to FIG. 1, the new medical device may be an integrated solution with two components: the patient side component and the central server-side component. The patient side component may include the Hdp monitoring system connected to the programmable generator, both connected to the processing system in the integrated hardware to make synchronization and compatible data aggregation. The central server-side component is connected with the patient side component by protected web-based platform and provides artificial intelligence-based computation and data storage.

Referring to FIG. 1, the two components of the new medical device related to the invention are real-time connected to bidirectional data transfer as described above. The programmable generator once loaded with control programs of selected series of SFq can be disconnected from the integrated solution in the invention for outpatient use. The programmable generator can be reconnected in the integrated solution in the invention for batch upload of update data and automatic treatment profiling transfer back to the processing system.

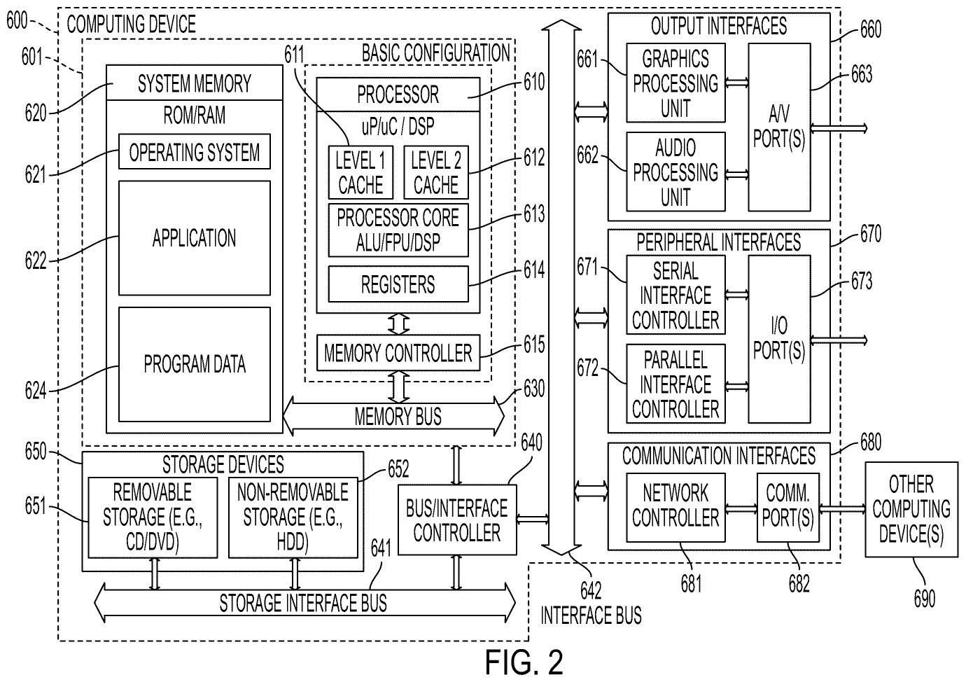

The system 11 includes an electrically conductive applicator 13 for applying one or more electromagnetic emissions to the warm-blooded mammalian subject. There are a number of different forms of applicators that may consist of an electrically conductive probe 13, which has a close contact with a subject undergoing treatment. Probe 13 is connected to an electromagnetic energy emitter (see also FIG. 2), through coaxial cable 12 and impedance matching transformer 14.

Electronic system 11 also includes a connector or coupler for connection to a programmable device such as a computer or an interface or receiver 16, which is adapted to receive an application storage device 52 such as, for example, magnetic media, semiconductor media, optical media, mechanically encoded media, or programmed emissions programmed with control information employed to control the operation of system 11 so that the desired type of low-energy emission therapy is applied to the patient.

Application storage device 52 can be provided with a microprocessor that, when applied to interface 16, operates to control the function of system 11 to apply the desired low-energy emission therapy. The application storage device 52 is provided with a microprocessor that is used in combination with microprocessor 21 within system 11. In such case, the microprocessor within device 52 assists in the interfacing of storage device 52 with system 11 and other central servers.

System 11 may also include a display 17 that can display various indications of the operation of system 11. In addition, system 11 may include on and off power buttons, optionally replaced by user interface 21A (refer to FIG. 2).

FIG. 2 depicts an illustrative block diagram of an Hdp monitoring system according to an embodiment. The system includes a computing device 600. The computing device may include various additional components such as basic configuration 601, a bus/interface controller 640, storage devices 650, output interfaces 660, peripheral interfaces 670, communication interfaces 680, and/or other computing devices 690. One or more electrical busses may be configured to operably connect the above-identified components. For example, storage interface bus 641 may be configured to operably connect the storage devices 650 and the bus/interface controller 640. Additionally, an interface bus 642 may be configured to operably connect the bus/interface controller 640 with the output interfaces 660, the peripheral interfaces 670, and the communication interfaces 680.

The basic configuration 601 may include a processor 610, a system memory 620, and a memory bus 630 configured to operably connect the processor and the system memory. In some examples, the processor 610 may include a level 1 cache 611, a level 2 cache 612, a processor core 613, one or more registers 614, and a memory controller 615. In some implementations, the system memory 620 may include various software or operating modules such as an operating system 621, one or more applications 622, and program data 624.

In some examples, the storage devices 650 may include a removable storage device 651 including, for example, a USB storage device or other similar removable media. The storage devices 650 may also include a non-removable storage device 652; such as a hard disk drive. In some implementations, the output interfaces 660 may include a graphics processing unit 661, an audio processing unit 662, and one or more A/V ports 663 operably connected to the graphics processing unit and the audio processing unit. In some examples, an external output device such as a monitor or other similar display and/or a speaker or other similar audio output device can be operably connected to the A/V ports 663.

In some examples, the peripheral interfaces 670 may include a serial interface controller 671, a parallel interface controller 672, and one or more I/O ports 673 operably connected to the serial interface controller 671 and the parallel interface controller 672. In some examples, an external device such as a printing device may be operably connected to the computing device 600 via the one or more I/O ports 673. In some implementations, the communication interfaces 680 may include a network controller 681 configured to facilitate communication with the other communication devices 690. In some examples, the network controller 681 may be operably connected to one or more communication ports 682 for establishing communications with the other communication devices 690. For example, the established communications can be via a wired or wireless data communication link.

In some implementations, the system as illustrated in FIG. 2 may be configured to measure Hdp values before, during, and/or after application of electromagnetic output signals to a patient. In an embodiment, circuitry may be provided with a connector configured to connect with the Hdp monitoring system. Alternatively, the circuitry may be integrated into the Hdp monitoring system. Descriptions of each of the blocks of the block diagram or functions thereof are included to facilitate an understanding thereof.

The block diagram of electronic circuitry of the Hdp monitoring system applies AM RF output signals to a patient at predetermined selected AM frequencies. The predetermined selected frequencies are controlled by AM frequency values stored in the storage device 52 and/or other servers. Various predetermined selected AM frequencies applied to a patient are indicated for treatment of patients suffering from a poor health condition for which the patient has been diagnosed.

In an embodiment, an integrated or combined device may enable sensing of Hdp values of a patient prior to, during, or after application of AM RF electromagnetic signals or other such signals. Of particular interest in this regard is that the measured and recorded Hdp values may differ dependent upon the patient condition. For example, measured and recorded Hdp values may differ among patients suffering from different forms of cancer. In addition, the measured and recorded Hdp values may differ from patients suffering from a form of cancer and healthy patients. However, such Hdp values may be substantially similar for patients suffering from the same or a closely related poor health condition. The measured and recorded representative Hdp variation values and the identification of SFq accordingly offer diagnosis and treatment opportunities for various forms of poor health conditions. In addition, such Hdp variation values may permit the diagnosis of healthy patient conditions.

Referring back to FIG. 1, here describing exclusively the programmed generator portion of the invention, the microprocessor 21 operates as the controller for the application system and is connected to control the various components of the system through address bus 22, data bus 23, and input/output (I/O) lines 25. Microprocessor 21 preferably includes internal storage for the operation code, control program, and temporary data. In addition, microprocessor 21 includes input/output (I/O) ports and internal timers. Microprocessor 21 may be, for example, an 8-bit single-chip micro-controller, 8048 or 8051 available from Intel Corporation of Santa Clara, California. The timing for microprocessor 21 is provided by a system clock, which includes a clock crystal along with capacitors. The system clock may run at any clock frequency suitable for the particular type of microprocessor used. In accordance with one embodiment, system clock 24 operates at a clock frequency of 8.0 MHz.

In general, microprocessor 21 functions to control controllable electromagnetic energy generator circuit 29 to produce a desired form of modulated low-energy electromagnetic emission for application to a patient through probe 13. Controllable generator circuit 29 includes modulation frequency generator circuit 31 and RF carrier signal oscillator 32. Microprocessor 21 operates to activate or de-activate controllable generator circuit 29 through oscillator disable line 33. Controllable generator circuit 29 also includes an AM modulator and power generator 34 that operates to amplitude modulate a carrier signal produced by RF carrier oscillator 32 on carrier signal line 36, with a modulation signal produced by modulation signal generator circuit 31 on modulation signal line 37. Modulator 34 produces an amplitude modulated carrier signal on modulated carrier signal line 38, which is then applied to the emitter output filter 39. The emitter output filter 39 is connected to probe 13 via coaxial line 12 and impedance transformer 14.

Microprocessor 21 controls modulation signal generator circuit 31 of controllable generator circuit 29 through address bus 22, data bus 23, and I/O lines 25. In particular, microprocessor 21 selects the desired waveform stored in a modulation waveform storage device via I/O lines 25. Microprocessor 21 also controls a waveform address generator to produce on a waveform address bus a sequence of addresses that are applied to modulation signal storage device 43 in order to retrieve the selected modulation signal. The desired modulation signal is retrieved from wave form look-up table 43 and applied to modulation signal bus 44 in digital form. Modulation signal bus 44 is applied to digital to analog converter (DAC) 46 which converts the digital modulation signal into analog form. This analog modulation signal is then applied to selective filter 47, which, under control of microprocessor 21, filters the analog modulation signal by use of a variable filter network including resistors and/or capacitors in order to smooth the wave form produced by the DAC on a modulation signal line.

In the present embodiment, the various modulation signal wave forms are stored in look-up table 43. In an embodiment, a look-up table 43 may contain up to 8 different modulation signal wave forms, although more or fewer may be stored in the lookup table. Wave forms that have been successfully employed include square wave forms or sinusoidal wave forms. Other possible modulation signal wave forms include rectified sinusoidal, triangular, and combinations of all of the above.

In an embodiment, each modulation signal wave form uses 256 bytes of memory and is retrieved from look-up table 43 by running through the 256 consecutive addresses. It is noted that each wave form may use more or fewer bytes of memory within the scope of this disclosure as will be apparent to one of ordinary skill in the related art. The frequency of the modulation signal is controlled by how fast the wave form is retrieved from look-up table 43. In an embodiment, this is accomplished by downloading a control code from microprocessor 21 into programmable counters contained within wave form address generator 41. The output of the programmable counters then drives a ripple counter that generates the sequence of addresses on the wave form address bus 42.

Waveform address generator 41 may, for example, be a programmable timer/counter uPD65042C, available from NEC. Modulation signal storage device or look-up table 43 may, for example, be a type 28C16 Electrical Erasable Programmable Read Only Memory (EEPROM) programmed with the desired wave form table. Digital to analog converter 46 may, for example, be a DAC port, such as AD557JN available from Analog Devices, and selective filter 47 may be a type 4052 multiplexer available from National Semiconductor or Harris Semiconductor. Additional or alternate components may be used within the scope of this disclosure.

The particular modulation control information used by microprocessor 21 to control the operation of controllable generator circuit 29, in accordance with the present invention, is stored in application storage device 52 or, in terms of the present invention may be a variable AM frequency tuning device adapted to load the interface 16 with AM frequencies between and high and low frequency levels. Application storage device 52 may be any storage device capable of storing information for later retrieval. Application storage device 52 is connected to the processing system by interface 16 to complete the integrated solution in the invention.

It should be emphasized that although the Figures illustrate microprocessor 21 separate from application storage device 52, microprocessor 21, and application storage device 52 where loaded control programs from the processing system are stored into the programmable generator. The control programs once loaded into the system control the operation of the system as described herein. In this case, interface 16 would exist between the combination of microprocessor 21 and application storage device 52 and the rest of the system.

Interface 16 is configured as appropriate for the particular application storage device 52 in use. Interface 16 translates the control information stored in application storage device 52 into a usable form for storage within the memory of microprocessor 21 to enable microprocessor 21 to control controllable generator circuit 29 to produce the desired modulated low-energy emission. Interface 16 may directly read the information stored on application storage device 52, or it may read the information through communication link with the processing system. When application storage device 52 and microprocessor 21 are incorporated in the same device, interface 16 is configured to connect microprocessor 21 to the rest of system.

The control information stored in application storage device 52 specifies various controllable parameters of the modulated low-energy RF electromagnetic emission which is applied to a patient through probe 13. Such controllable parameters include, for example, the frequency and amplitude of the carrier, the amplitudes and frequencies of the modulation of the carrier, the duration of the emission, the power level of the emission, the duty cycle of the emission (i.e., the ratio of on time to off time of pulsed emissions applied during an application), the sequence of application of different modulation frequencies for a particular application, and the total number of treatments and duration of each treatment prescribed for a particular patient.

For example, the carrier signal and modulation signal may be selected to drive the probe 13 with an amplitude modulated signal in which the carrier signal includes spectral frequency components below 1 GHz, and preferably between 1 MHz and 900 MHz, and in which the modulation signal comprises spectral frequency components between 0.1 Hz and 10 MHz, and preferably between 1 Hz and 150 KHz. In an embodiment, one or more modulation frequencies may be sequenced to form the modulation signal.

As an additional feature, an electromagnetic emission sensor 53 may be provided to detect the presence of electromagnetic emissions at the frequency of the carrier oscillator 32. Emission sensor 53 provides to microprocessor 21 an indication of whether or not electromagnetic emissions at the desired frequency are present. Microprocessor 21 then takes appropriate action, for example, displaying an error message on information output display 17, disabling controllable generator circuit 29, or the like.

The system may further include a power sensor 54, which detects the amount of power applied to the patient through probe 13 compared to the amount of power returned or reflected from the patient. This ratio is indicative of the proper use of the system during a therapeutic session. Power sensor 54 applies to microprocessor 21 through power sense line 56 an indication of the amount of power applied to patient through probe 13 relative to the amount of power reflected from the patient.

The indication provided on power sense line 56 may be digitized and used by microprocessor 21, for example, to detect and control a level of applied power, and to record on application storage device 52, information related to the actual treatments applied. Data transfer information to the processing system may include, for example: the number of treatments applied for a given time period; the actual time and date of each treatment; the number of attempted treatments; the treatment compliance (i.e., whether the probe was in place or not in place during the treatment session); and the cumulative dose of a particular modulation frequency.

The level of power applied is preferably controlled to cause the specific absorption rate (SAR) of energy absorbed by the patient to be from 1 microwatt per kilogram of tissue to 50 Watts per kilogram of tissue. Preferably, the power level is controlled to cause an SAR of from 100 microwatts per kilogram of tissue to 10 Watts per kilogram of tissue. Most preferably, the power level is controlled to deliver whole body mean SAR in the range of only 0.2 to 1 mW/kg, with a 1 g peak spatial SAR between 150 and 350 mW/kg. These SARs may be in any tissue of the patient. The system also includes powering circuitry including battery and charger circuit 57 and battery voltage change detector 58.

In the integrated solution, combination or use of two medical devices of the nature described above but executing different and synchronized tasks that derivate from their initial conception and application, such as measuring and recording Hdp values, at least nine parameter values mentioned, before, during, or after exposure or application of EMF frequency output signals and identifying representative Hdp variation values and SFq have provided a scientific and reproducible method of diagnosing and treating health conditions of a patient, further scientific details related, for example, specifically to one different form of cancer diagnoses are provided below:

The identification of changes in pulse amplitude in patients with a diagnosis of cancer when exposed to low and safe levels of 27.12 MHz radiofrequency electromagnetic fields amplitude-modulated at specific frequencies have previously been reported. (Barbault, A. et al., Amplitude-modulated electromagnetic fields for the treatment of cancer: discovery of tumor-specific frequencies and assessment of a novel therapeutic approach, J. Exp. Clin. Cancer Res. 28, 51, doi:10.1186/1756-9966-28-51 (2009)). The observation that changes in pulse amplitude occur at exactly the same frequencies in patients with the same type of cancer led to a hypothesis that each type of cancer possesses a specific frequency signature. (Id.) In vitro experiments have shown that tumor-specific frequencies have anti-proliferative effects on cancer cells, modulate the expression of genes involved in cell migration and invasion, and are capable of disrupting the mitotic spindle. (Zimmerman, J. W. et al., Cancer cell proliferation is inhibited by specific modulation frequencies, British Journal of Cancer 106, 307-313 (2012)). The clinical activity of these tumor-specific frequencies was assessed in two separate studies in which patients were treated with intrabuccally administered AM RF EMF, which were modulated at tumor-specific frequencies. Antitumor activity was observed in patients with metastatic breast cancer (Barbault, A. et al. (2009)) and advanced hepatocellular carcinoma (Costa, F. P. et al., Treatment of advanced hepatocellular carcinoma with very low levels of amplitude-modulated electromagnetic fields, British Journal of Cancer 105, 640-648 (2011)) and stable disease was observed in patients with other tumor types.

This study was designed to test the hypothesis that analysis of changes in Hdp upon exposure to tumor-specific frequencies is a novel, non-invasive diagnostic approach in warm-blooded mammalian subjects. The method is also capable of identifying SFq to be used in the treatment of health condition in patients.

The experimental procedures described below were reviewed and approved by the Hospital Sírio Libanês Institutional Review Board (IRB), Rua Dona Adma Jafet,50 Conj.41/43, São Paulo S P 01.308-050 Brazil. All patients and healthy individuals enrolled in this study signed an informed consent, which was approved by the IRB. The protocol was registered prior to enrolment of the 1st patient: clinicaltrial.gov identified no. NCT 01686412. 87 individuals were screened and 82 individuals were prospectively enrolled. The patient's diagnosis and the nature of AM RF EMF exposure (HCC-specific, breast cancer specific, and randomly chosen frequencies) were disclosed before computational analysis in order to constitute a knowledge base. The validation group included patients with biopsy-proven cancer (advanced HCC and advanced breast cancer) and healthy controls. The last group included patients with potentially resectable HCC.

The AM RF EMF device used for this study has been described in detail previously. (Costa, F. P. et al., British journal of cancer 105, 640-648 (2011)). While patients receiving treatment with AM RF EMF are exposed to three, daily one hour treatments, the diagnostic feasibility of AM RF EMF administration was tested during a single 10 minute exposure in order to expose all individuals once to each of the 194 tumor-specific frequencies (HCC specific and breast cancer specific), which are each emitted for three seconds. (Barbault, A. et al., J. Exp. Clin. Cancer Res. 28, 51, doi: 10.1186/1756-9966-28-51 (2009); Zimmerman, J. W. et al., British Journal of Cancer 106, 307-313 (2012); Costa, F. P. et al., British Journal of Cancer 105, 640-648 (2011)). Similarly, 194 of the previously reported 236 randomly chosen frequencies were selected (Zimmerman, J. W. et al., British Journal of Cancer 106, 307-313 (2012)) to match the number and exposure duration of tumor-specific frequencies. Hence, each individual was exposed to all frequencies included in each of the treatment programs (HCC specific, breast cancer specific, and randomly chosen frequencies). Each modulation frequency was emitted for three seconds from the lowest to the highest frequency as previously described. ((Barbault, A. et al., J. Exp. Clin. Cancer Res. 28, 51, doi: 10.1186/1756-9966-28-51 (2009); Zimmerman, J. W. et al., British journal of cancer 106, 307-313 (2012); Costa, F. P. et al., British journal of cancer 105, 640-648 (2011)).

The following Examples are provided solely for illustrative and exemplary purposes, and are not intended to limit the invention in any way.

### Example 1

Exemplary of the Hdp values recorded during 23 consecutive heart-beats are set forth below in TABLE 1. The measured and recorded Hdp values for each of the nine hemodynamic parameters are exemplary of such values exhibited by a single patient.

| | | | | | | | | | | | TPR | TPRI |
|---|---|---|---|---|---|---|---|---|---|---|---|---|
| Time | Beat | RRI | HR | sBP | dBP | mBP | SV | SI | CO | CI [l/ | [symbol] | [symbol] |
| [s] | [1] | [%] | [bpm] | [mmHg] | [mmHg] | [mmHg] | [ml] | [ml/m²] | [l/min] | (min*m²)] | [symbol] | [symbol] |
| 86.23 | 88 | 865.667 | 69.311 | 111.109 | 81.39 | 93.019 | 61.348 | 31.86 | 4.252 | 2.208 | 1693.647 | 3261.151 |
| 87.09 | 89 | 873.375 | 68.699 | 111.892 | 82.068 | 94.581 | 59.202 | 30.746 | 4.067 | 2.112 | 1801.402 | 3468.635 |
| 87.97 | 90 | 864.667 | 69.391 | 112.089 | 81.795 | 94.622 | 55.133 | 28.633 | 3.826 | 1.987 | 1915.923 | 3689.148 |
| 88.83 | 91 | 859.042 | 69.845 | 110.589 | 82.004 | 94.27 | 55.858 | 29.009 | 3.901 | 2.026 | 1871.508 | 3603.625 |
| 89.69 | 92 | 842.708 | 71.199 | 110.359 | 79.861 | 93.377 | 53.523 | 27.797 | 3.811 | 1.979 | 1897.299 | 3653.285 |
| 90.53 | 93 | 857.417 | 69.978 | 109.608 | 81.868 | 92.172 | 55.005 | 28.587 | 3.849 | 1.999 | 1853.334 | 3568.631 |
| 91.39 | 94 | 876.375 | 68.464 | 110.711 | 81.306 | 93.933 | 58.008 | 30.126 | 3.971 | 2.063 | 1831.749 | 3527.069 |
| 92.27 | 95 | 864 | 69.444 | 111.357 | 81.383 | 94.184 | 54.844 | 28.483 | 3.809 | 1.978 | 1915.33 | 3688.006 |
| 93.13 | 96 | 858.333 | 69.903 | 109.975 | 80.985 | 93.399 | 55.252 | 28.695 | 3.862 | 2.006 | 1872.449 | 3605.438 |
| 93.99 | 97 | 851.75 | 70.443 | 108.478 | 79.125 | 92.46 | 53.177 | 27.617 | 3.746 | 1.945 | 1910.548 | 3678.798 |
| 94.84 | 98 | 847 | 70.838 | 107.625 | 79.9 | 90.667 | 54.014 | 28.051 | 3.826 | 1.987 | 1832.962 | 3529.405 |
| 95.69 | 99 | 870.333 | 68.939 | 108.472 | 81.437 | 92.267 | 59.124 | 30.705 | 4.076 | 2.117 | 1752.081 | 3373.666 |
| 96.56 | 100 | 866.75 | 69.224 | 110.854 | 80.602 | 93.625 | 56.905 | 29.553 | 3.939 | 2.046 | 1840.477 | 3543.875 |
| 97.42 | 101 | 857.667 | 69.957 | 109.651 | 80.548 | 93.082 | 59.392 | 30.845 | 4.155 | 2.158 | 1734.467 | 3339.75 |
| 98.28 | 102 | 852.042 | 70.419 | 108.832 | 79.499 | 92.771 | 56.965 | 29.584 | 4.011 | 2.083 | 1790.329 | 3447.314 |
| 99.13 | 103 | 844.042 | 71.087 | 108.254 | 79.411 | 91.44 | 55.587 | 28.868 | 3.951 | 2.052 | 1790.527 | 3447.694 |
| 99.98 | 104 | 862.375 | 69.575 | 105.461 | 80.022 | 89.712 | 58.751 | 30.512 | 4.088 | 2.123 | 1697.054 | 3267.711 |
| 100.84 | 105 | 866 | 69.284 | 107.714 | 79.491 | 91.673 | 57.207 | 29.71 | 3.964 | 2.058 | 1789.787 | 3446.271 |
| 101.71 | 106 | 858 | 69.93 | 107.364 | 79.047 | 91.513 | 54.291 | 28.195 | 3.797 | 1.972 | 1865.124 | 3591.332 |
| 102.56 | 107 | 853.333 | 70.313 | 106 | 79.964 | 91.438 | 51.111 | 28.544 | 3.594 | 1.866 | 1988.712 | 3790.793 |
| 103.42 | 108 | 847.75 | 70.776 | 107.409 | 78.102 | 91.28 | 54.451 | 28.279 | 3.854 | 2.001 | 1832.565 | 3528.64 |
| 104.26 | 109 | 847.667 | 70.783 | 106.429 | 78.788 | 89.288 | 55.211 | 28.673 | 3.908 | 2.03 | 1766.407 | 3401.251 |
| 105.11 | 110 | 871.375 | 68.857 | 106.965 | 79.41 | 90.751 | 59.018 | 30.651 | 4.064 | 2.11 | 1727.465 | 3326.267 |

[symbol]

indicates data missing or illegible when filed

Exemplary of the dependent new attributes parameters values recorded during 23 consecutive heart-beats are set forth below in TABLE 2. The dependent new attributes parameters values may be used as representative Hbp variation values exhibited by a single patient.

| | | | RRISD1 × | | | | |
|---|---|---|---|---|---|---|---|
| Beat | RRISD1 | RRISD2 | SD2 | RRICCM | RRITPVa | Frequency | DI |
| 1 | 13.8789264 | 53.7068018 | 745.392747 | −6907.8288 | 57.3037533 | 4 | 8.55E+07 |
| 2 | 51.6567542 | 84.9515646 | 4388.32209 | −6935.7519 | 89.7056474 | 14 | 8.58E+07 |
| 3 | 27.8759158 | 40.6380658 | 1132.8233 | 11809.1202 | 43.0042157 | 24 | 1.46E+08 |
| 4 | 40.5454393 | 34.3807524 | 1393.98271 | −26582.556 | 36.3831032 | 34 | 3.29E+08 |
| 5 | 35.0480871 | 40.5817451 | 1422.31253 | −3496.0755 | 42.8842484 | 44 | 4.32E+07 |
| 6 | 42.5039809 | 29.4086701 | 1249.98555 | 990.223308 | 30.8444667 | 54 | 1.24E+07 |
| 7 | 45.1901092 | 36.0755196 | 1630.25667 | 3137.92983 | 38.4382246 | 64 | 3.90E+07 |
| 8 | 46.765476 | 33.6755254 | 1574.85198 | −22828.727 | 35.9011603 | 74 | 2.83E+08 |
| 9 | 39.8256199 | 32.1894788 | 1281.96595 | 47.5721464 | 34.3384147 | 84 | 1.78E+06 |
| 10 | 30.2713377 | 47.7661583 | 1445.94551 | 4519.26636 | 50.6761039 | 94 | 5.61E+07 |
| 11 | 35.186602 | 23.8825096 | 840.344361 | 2685.20061 | 25.1495254 | 104 | 3.33E+07 |
| 12 | 37.479101 | 52.3296708 | 1961.26902 | −18712.683 | 55.6259921 | 114 | 2.32E+08 |
| 13 | 53.6928877 | 42.3805183 | 2275.53241 | 4844.24643 | 44.7894832 | 124 | 6.02E+07 |
| 14 | 34.0572425 | 44.5420995 | 1516.98109 | −11106.596 | 47.2445818 | 134 | 1.37E+08 |
| 15 | 46.6213262 | 24.6540229 | 1149.40324 | 12824.1034 | 26.2531618 | 144 | 1.59E+08 |
| 16 | 32.445878 | 39.6020208 | 1284.92233 | −30406.893 | 41.9408539 | 154 | 3.77E+08 |
| 17 | 74.9106161 | 98.5793958 | 7384.64327 | −13693.11 | 104.217338 | 164 | 1.69E+08 |
| 18 | 44.7166188 | 40.2697042 | 1800.72501 | −11245.794 | 42.4305224 | 174 | 1.39E+08 |
| 19 | 49.3476794 | 49.535279 | 2444.45107 | −1585.6643 | 52.3216238 | 184 | 1.97E+07 |
| 20 | 34.0536542 | 34.4127831 | 1171.88101 | 4490.32455 | 36.3052018 | 194 | 5.57E+07 |
| 21 | 35.2361001 | 23.5102587 | 828.409828 | −11087.768 | 24.8419718 | 204 | 1.37E+08 |
| 22 | 37.1406638 | 54.5350674 | 2025.46861 | 9562.95019 | 57.4914902 | 214 | 1.19E+08 |
| 23 | 32.5973769 | 42.9152428 | 1398.92435 | −11403.819 | 45.5420335 | 224 | 1.41E+08 |

### Example 2

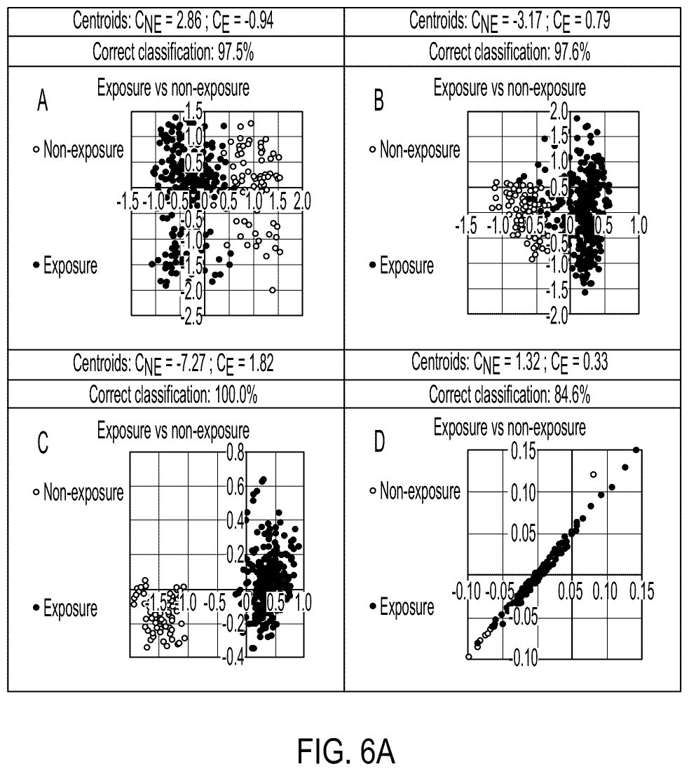

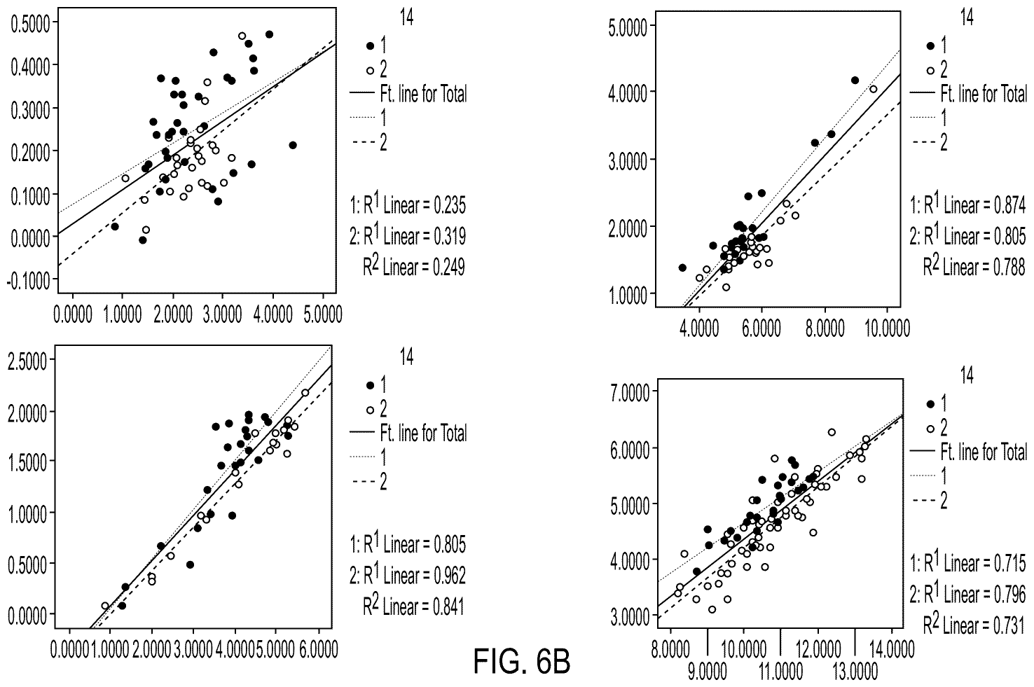

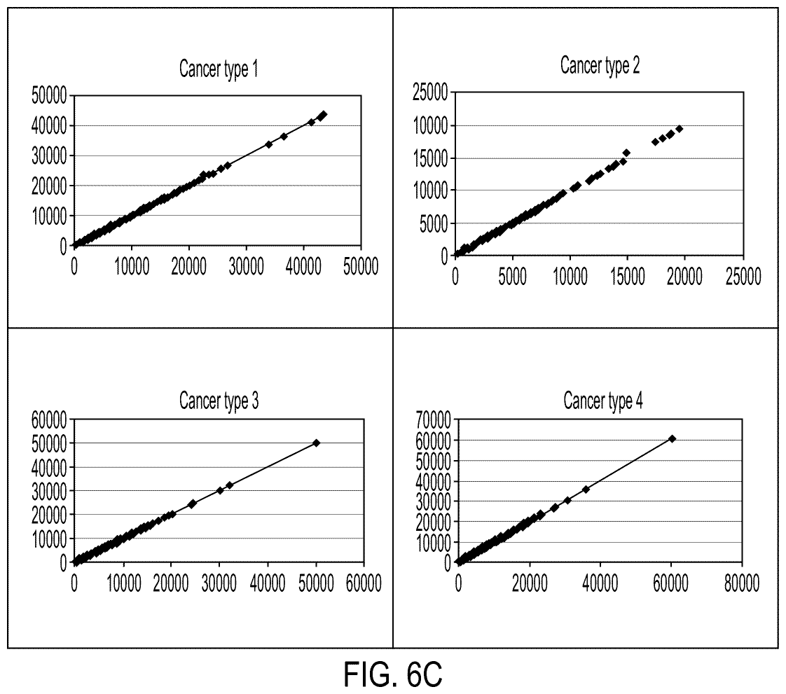

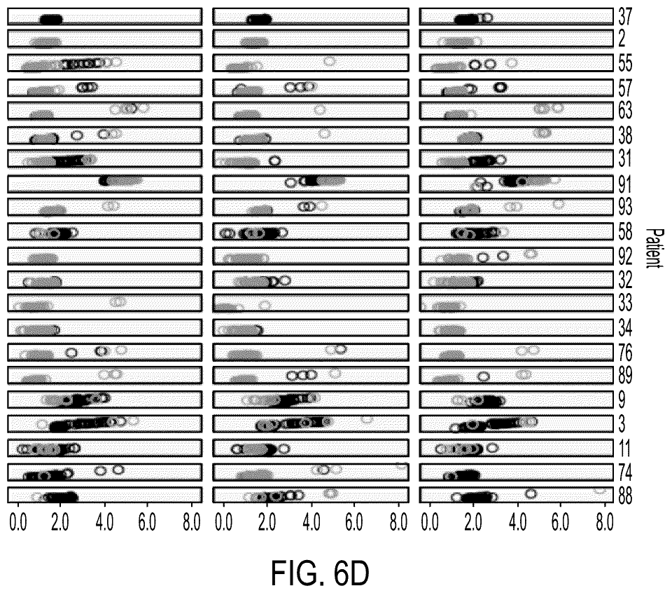

Referring now to FIG. 6A, there are a series of plots of Hdp variation values from four different patients diagnosed with hepatocellular carcinoma shown. FIG. 6 demonstrates high rates of correct classification using centroids from representative Hdp variation values during basal Hdp values (white) and exposure Hdp values (dark) in four patients with a diagnosis of hepatocellular carcinoma during systemic treatment. Patients A, B, and C were not responding to the cancer treatment, and Patient D was experiencing good response to cancer treatment. The centroids from representative Hdp variation values patterns were significantly different between exposure versus non-exposure periods and responding versus non-responding to treatment using four different well-established statistical methods (p<0.0001).

### Example 3

Referring now to FIG. 6B, there are a series of plots of physiological responses in responses to reactive and non-reactive pulses for four patients with diagnosis of hepatocellular carcinoma shown. FIG. 7 demonstrates high rates of correlation of representative Hdp variation values and a bio-feedback procedure involving very substantial observations and measurements of physiological responses (at certain well defined AM frequencies) for reactive pulse (e.g., in black) and non-reactive pulse (e.g., in white) in four patients with diagnosis of hepatocellular carcinoma. The representative Hdp variation values patterns were significantly different between reactive versus non-reactive pulse alterations using different well-established statistical methods (p<0.0001).

### Example 4

Differences in representative Hdp variation values are determined during the exposure to different SFq. The application of mathematical algorithms and artificial intelligence processing can be used to identify EMF frequencies that may cause Hdp variation value changes consistent with a healthy condition of a patient, which are referred to as SFq. Some example SFq experimentally identified for a set of patients are set forth below in TABLE 3.

| Patient code | frequency code | SD1 | SD2 | DISD1 | DISD2 |
|---|---|---|---|---|---|
| 4 | 4 | 13.8789264 | 53.7068018 | 27.530306 | 7.66375275 |
| 4 | 14 | 51.6567542 | 84.9515646 | 10.2475218 | 23.5810101 |
| 4 | 24 | 27.8759158 | 40.6380658 | 13.5333166 | 20.7324887 |
| 4 | 34 | 40.5454393 | 34.3807524 | 0.86379312 | 26.9898021 |
| 4 | 44 | 35.0480871 | 40.5817451 | 6.36114534 | 20.7888095 |
| 4 | 54 | 42.5039809 | 29.4086701 | 1.09474849 | 31.9618844 |
| 4 | 64 | 45.1901092 | 36.0755196 | 3.78087676 | 25.2950349 |
| 4 | 74 | 46.765476 | 33.6755254 | 5.35624362 | 27.6950291 |
| 4 | 84 | 39.8256199 | 32.1894788 | 1.58361254 | 29.1810757 |
| 4 | 94 | 30.2713377 | 47.7661583 | 11.1378947 | 13.6043962 |
| 4 | 104 | 35.186602 | 23.8825096 | 6.22263042 | 37.4880449 |
| 4 | 114 | 37.479101 | 52.3296708 | 3.93013139 | 9.04088375 |
| 4 | 124 | 53.6928877 | 42.3805183 | 12.2836553 | 18.9900362 |
| 4 | 134 | 34.0572425 | 44.5420995 | 7.35198986 | 16.828455 |
| 4 | 144 | 46.6213262 | 24.6540229 | 5.21209376 | 36.7165316 |
| 4 | 154 | 32.445878 | 39.6020208 | 8.9633544 | 21.7685338 |
| 4 | 164 | 74.9106161 | 98.5793958 | 33.5013837 | 37.2088413 |
| 4 | 174 | 44.7166188 | 40.2697042 | 3.3073864 | 21.1008503 |
| 4 | 184 | 49.3476794 | 49.535279 | 7.93844703 | 11.8352755 |
| 4 | 194 | 34.0536542 | 34.4127831 | 7.35557819 | 26.9577715 |

### Example 5

Referring now to FIG. 6C, there are shown plots demonstrating that different health condition-specific SFq can be defined by linear relationships. Health condition-specific SFq from amplitude modulated from 100 Hz to 40,000 Hz are distributed in an apparent chaotic manner in different health conditions. By reorganizing different health condition-specific SFq using mathematical calculations described in the invention, we obtain linear equation defined by Hz=a+βx with R²=99.9%. The artificial intelligence algorithms allow the construction of series of SFq for the diagnosis and treatment of patients. The example showed the distribution of 1,054 cancer-specific frequencies from four cancer types.

Referring now to FIG. 6D, health condition-specific SFq from 100 Hz to 40,000 Hz are distributed in a determined manner in different health conditions. The artificial intelligence algorithms allow the identification of patterns in series of SFq used the diagnosis of patients. FIG. 6D shows the distribution of disease-specific SFq (gray or light) and healthy-specific SFq (dark) during the exposure to three different groups of cancer-specific frequencies in 21 patients.

### Example 6

A Phase I Study of Therapeutic Amplitude-Modulated Electromagnetic Fields in Advanced Tumors by Boris Pasche, Alexandre Barbault, Brad Bottger, Fin Bomholt, and Niels Kuster.

### Background

In vitro studies suggest that low levels of amplitude-modulated electromagnetic fields may modify cell growth. Specific frequencies have been identified, specific frequencies that may block cancer cell growth. A portable and programmable device capable of delivering low levels of amplitude-modulated electromagnetic fields has been developed. The device emits a 27.12 MHz radiofrequency signal, amplitude-modulated at cancer-specific frequencies ranging from 0.2 to 23,000 Hz with high precision. The device is connected to a spoon-like coupler, which is placed in the patient's mouth during treatment.

### Methods

A phase I study was conducted consisting of three daily 40 min treatments. From March 2004 to September 2006, 24 patients with advanced solid tumors were enrolled. The median age was 57.0+/−12.2 years. 16 patients were female. As of January 2007, 5 patients are still on therapy, 13 patients died of tumor progression, two patients are lost to follow-up and one patient withdrew consent. The most common tumor types were breast (7), ovary (5) and pancreas (3). 22 patients had received prior systemic therapy and 16 had documented tumor progression prior to study entry.

### Results

The median duration of therapy was 15.7+/−19.9 weeks (range: 0.4-72.0 weeks). There were no NCI grade 2, 3, or 4 toxicities. Three patients experienced grade 1 fatigue during and immediately after treatment. 12 patients reported severe pain prior to study entry. Two of them reported significant pain relief with the treatment. Objective response could be assessed in 13 patients, six of whom also had elevated tumor markers. Six additional patients could only be assessed by tumor markers. Among patients with progressive disease at study entry, one had a partial response for >14.4 weeks associated with >50% decrease in CEA, CA 125 and CA 15-3 (previously untreated metastatic breast cancer); one patient had stable disease for 34.6 weeks (add info); one patient had a 50% decrease in CA 19-9 for 12.4 weeks (recurrent pancreatic cancer). Among patients with stable disease at enrollment, four patients maintained stable disease for 17.0, >19.4, 30.4, and >63.4 weeks.

### Conclusions

The treatment is a safe and promising novel treatment modality for advanced cancer. A phase II study and molecular studies are ongoing to confirm those results.

### Example 7

A Phase II Study of Therapeutic Amplitude-Modulated Electromagnetic Fields in the Treatment of Advanced Hepatocellular Carcinoma (HCC) Federico P Costa, Andre Cosme de Oliveira, Roberto Meirelles Jr., Rodrigo Surjan, Tatiana Zanesco, Maria Cristina Chammas, Alexandre Barbault, Boris Pasche.

### BACKGROUND

Phase I data suggest that low levels of electromagnetic fields amplitude-modulated at specific frequencies administered intrabuccally with the device of Example A are a safe and potentially effective treatment for advanced cancer. The device emits a 27.12 MHz RF signal, amplitude-modulated with cancer-specific frequencies ranging from 0.2 to 23,000 Hz with high precision. The device is connected to a spoon-like coupler placed in the patient's mouth during treatment. Patients with advanced hepatocellular carcinoma HCC and limited therapeutic options were offered treatment with a combination of HCC-specific frequencies.

### Methods

From October 2005 to July 2007, 43 patients with advanced HCC were recruited in a phase II study. Two patients were considered screening-failures. The patients received three, daily 1 hour treatments until disease progression or death. The median age was 64.0+/−14.2 years. 17 patients were Child-Pugh status A5-6, and 24 patients were Child-Pugh B7-9. 75.6% of the patients had documented progression of disease (POD) prior to study entry.

### Results

The overall objective response rate as defined by partial response (PR) or stable disease (SD) in patients with documented POD at study entry; 4 PR (1 with near complete response for 58 months) and 16 SD. The median survival was 6.7 months (95% CI 3.0-10.2) and median progression-free survival of 4.4 months (95% 2.1-5.3). 14 patients have received therapy for more than six months. The estimated survival at 12, 24, and 36 months were 27.9%, 15.2% and 10.1% respectively. 12 patients reported pain at study entry: eight of them (66%) experienced decreased pain during treatment. There were no NCI grade 2/3/4 toxicities. One patient developed grade 1 mucositis and grade 1 fatigue.

### Conclusion

In patients with advanced HCC, the treatment is a safe and effective novel therapeutic option, which has antitumor effects and provides pain relief in the majority of patients.

Thus, it is seen that the electronic device of the present invention, comprising means for the accurate control over the frequencies and stability of amplitude modulations of a high frequency carrier signal, provides a safe and promising novel treatment modality for the treatment of patients suffering from various types of advanced forms of cancer.

Exemplary of above accurately controlled amplitude modulated frequencies controlling the frequency of amplitude modulations of a high frequency carrier signal are set forth below along with the type of cancer or tumor harbored by a subject to be treated.

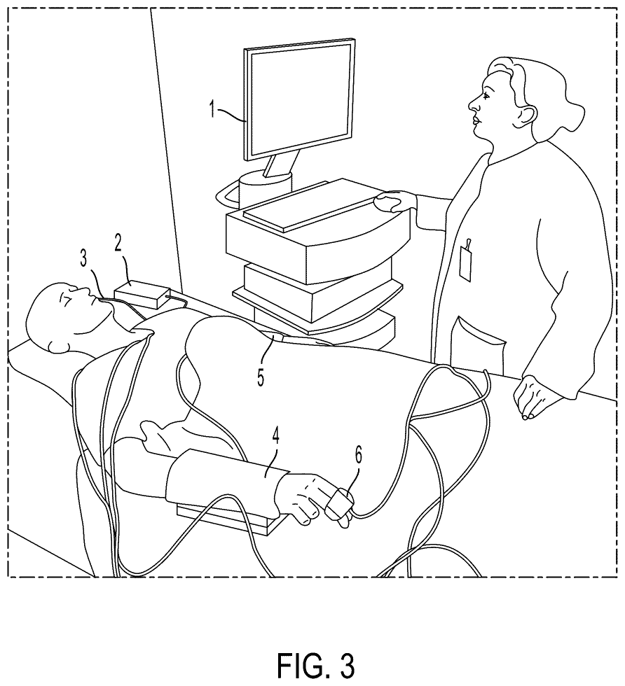

Referring again to the figures, FIG. 3 depicts an illustrative patient during experimental setup for continuous monitoring of hemodynamic parameters before and during AM RF EMF exposure. Non-invasive hemodynamic measurement was performed using a Task Force® Monitor (CNSystems Medizintechnik GmbH, version 2.2.12.0, Reininghausstraße 13, 8020 Graz, Austria). Numerical values of heart rate, blood pressure, and blood flow are measured by digital photoplethysmography, pressure cuff, and ECG. The hemodynamic parameters are transformed into absolute values for each consecutive heartbeat before and during AM RF EMF exposure. The spoon-shaped antenna for intrabuccal administration of AM RF EMF was placed in the patient's mouth during the entire experiment. In FIG. 3, the Task Force® monitor is labelled 1. The AM RF EMF emitting device is labelled 2. The AM RF EMF emitting device connected to a coaxial cable, which is connected to the spoon-shaped antenna 3. A right arm digital pressure cuff is labelled 4. A digital photoplethysmography is labelled 5. A left arm digital pressure cuff is labelled 6. Electrodes' cables for ECG and impedance cardiography are provided.

Numerical values of heart rate variability, blood pressure, baroreceptor sensitivity, and blood pressure were measured by digital photoplethysmography and ECG acquired through three thoracic adhesive electrodes for high resolution for RR interval analysis. Digital pressure cuff was placed on the right arm and around the middle phalanx of the third and fourth right finger and another on the left arm between the shoulder and the elbow. Blood pressure measurements were transformed into absolute values for each consecutive heartbeat.

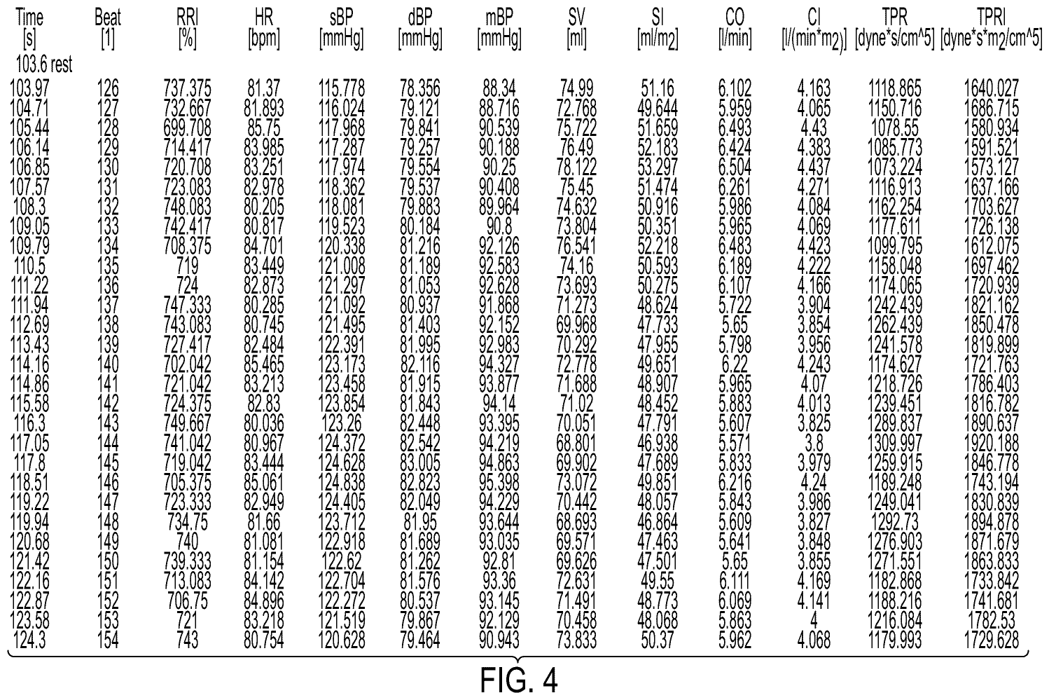

Referring to FIG. 4, an example is seen of eleven hemodynamic parameters simultaneously measured during each heartbeat: heart rate (HR), systolic blood pressure (sBP), median blood pressure (mBP), diastolic blood pressure (dBP), total peripheral resistance (TPR), total peripheral resistance index (TPRI), cardiac output (CO), cardiac index (CI), RR interval (RRI), stroke volume (SV), and systolic index (SI). Hemodynamic recording was performed continuously in supine position before and during exposure to AM RF EMF. A total of three million hemodynamic parameters were analyzed in this study.

Participants held the spoon-shaped antenna in their mouth during the entire experiment. The three different devices each programmed with one of the treatment programs (HCC specific, breast cancer specific, and randomly chosen frequencies) were connected prior to initiation of each of the AM RF EMF exposure period. The protocol was conducted in a double-blind fashion.

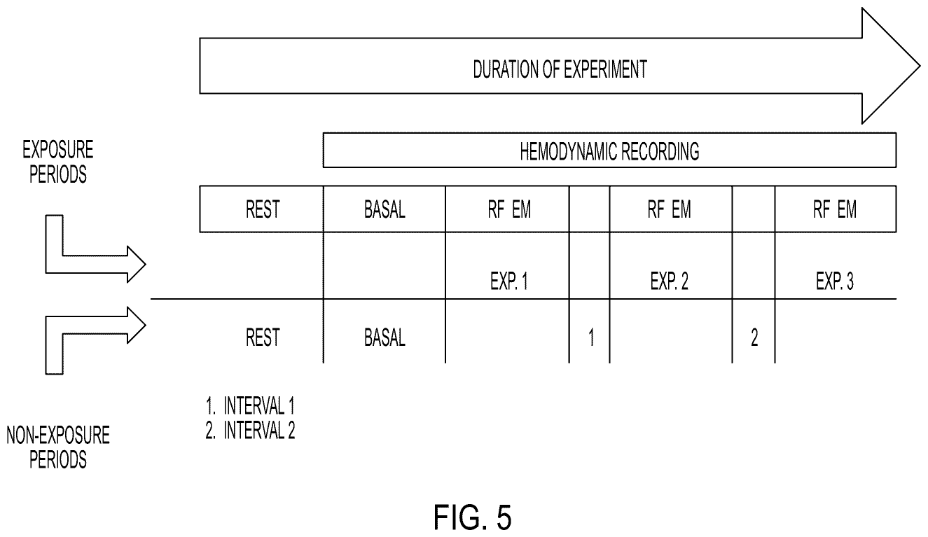

Referring to FIG. 5, hemodynamic recording was performed continuously during non-exposure and exposure periods in a double-blind fashion. The non-exposure periods were the initial basal and five-minute resting intervals between RF EMF exposures. During the exposure periods, patients received AM RF EMF (HCC-specific, breast cancer-specific, randomly chosen frequencies).

Hemodynamic parameters were analyzed according to three factors: diagnosis (HCC, breast cancer, healthy control), gender, and recording period (baseline and exposure to HCC-specific, breast cancer-specific, and randomly chosen modulation frequencies).

Analysis of the recorded hemodynamic data was only conducted after completion of patient accrual. Patients were selected to constitute a knowledge base for machine learning. The anticipated outcome of the knowledge base group analysis was the creation of computative specific for patients with hepatocellular carcinoma, patients with breast cancer, and healthy controls. Once the computations were constructed, the data from the validation group was analyzed in a blinded fashion in order to validate the computative.

Analysis of six patients diagnosed with potentially resectable HCC was included in the validation group analysis. These patients underwent the same non-invasive hemodynamic parameter measurements within 24 hours prior to HCC surgical resection and after complete recovery within four to six weeks post-surgery. Pre- vs post-surgical analysis was conducted.

Analysis of hemodynamic parameters during the basal non-exposure period was significantly different among healthy controls, patients with hepatocellular carcinoma, and patients with breast cancer in the “Discovery Group” (p<0.0001). There were significant differences in hemodynamic parameters between male and female participants as well.

Hemodynamic parameter analysis during the basal non-exposure and exposure periods were conducted separately. Differences of representative Hdp variation values are determined during the exposure to baseline and exposure to HCC-specific, breast cancer-specific, and randomly chosen modulation frequencies separated in different SFq. The application of mathematical algorithms and artificial intelligence processing identify HRV patterns for each different SFq for each individual being diagnosed by HCC, breast cancer, and healthy control.

The identification of HRV patterns with the application of mathematical algorithms and artificial intelligence processing resulting from the exposure to modulated frequencies in healthy controls and patients with cancer (HCC or Breast cancer) demonstrated differences in the HRV patterns that could be used in the identification of the patient's diagnosis.

Using a previously selected knowledge base group constituted by 10 patients with biopsy proven HCC and 10 male healthy controls, the artificial intelligent processing analyzed the 10 min baseline non-exposure period in combination of 582 modulated frequencies in order to identify specific HRV patterns used in the diagnosis of a patient.

A validation group of 40 male individuals was tested. The diagnosis was correctly identified in 37 individuals. There were 2 healthy controls labeled as HCC patients and one HCC patient labeled as healthy individual. The artificial intelligent processing algorithm also indicated relevant modulated frequencies or SFq used in the correct discrimination between HCC and healthy controls.

Using a previously selected knowledge base group constituted by 10 patients with biopsy proven breast cancer and 10 female healthy controls, the artificial intelligent processing analyzed the 10 min baseline non-exposure period in combination of 582 modulated frequencies in order to identify specific HRV patterns used in the diagnosis of a patient.

A validation group of 27 female individuals was tested. The diagnosis was correctly identified in 17 of 18 breast cancer patients. The artificial intelligent processing algorithm also indicated relevant modulated frequencies or SFq used in the correct discrimination between HCC and healthy controls.

The identity of specific HRV patterns for modulated frequencies predominantly observed in HCC that significantly differ from healthy controls are selected to be used in the treatment programs of patients with HCC as SFq. The same rational applies for breast cancer patients and possibly other healthy conditions.

In accordance with another aspect of the present invention, the identification and characterization of new methods allowing for the diagnosis of hepatocellular carcinoma and breast cancer in a blinded fashion based solely on the identification of HRV patterns during exposure to 27.12 MHz RF EMF amplitude modulated at tumor-specific frequencies are provided. These findings may have broad clinical implications for the diagnosis of cancer.

U.S. patent application Ser. No. 12/450,450 listed those frequencies known as of the filing date of 25 Sep. 2009, while U.S. Pat. No. 8,977,365 added those frequencies known as of its filing date of 22 Aug. 2012. Since those filings, additional AM frequencies have been determined by a bio-feedback procedure involving very substantial observations and measurements of physiological responses (at certain well defined AM frequencies) by subjects exposed to low-energy electromagnetic emission excitation have been determined to be efficacious in the characterization, diagnosis, treatment, and frequency discovery of the type of cancer or tumor harbored by a subject to be treated. Based on such multiple amplitude modulation frequency values it has been surprisingly discovered that a relationship exists between sequential values or sequential groups of values within the range of frequency values.

Given that SFq are linearly correlated to a series of numbers, which constitute a superset of the series of prime numbers, the construction of series of common denominators characterizing all SFq that have been previously patented and determined by a bio-feedback procedure involving very substantial observations and measurements of physiological responses (at certain well defined AM frequencies) by subjects exposed to low-energy electromagnetic emission excitation in any health condition of a patient is now being determined by a new the mathematical model described below as an important aspect of the invention in order to perform precise diagnosis and treatment of warm-blooded mammalian subjects.

SFq can be organized in seven infinite families of congruent elements (mod 7), using n=7x+i, where x=1, 2, . . . is a natural number and i=0, 1, 2, . . . , 6 is the family index or remainder. Allocating n in blocks b of i×k=42 positions, where k=1, 2, . . . , 6 defines the position of n in the family i in the respective block resulting, by construction, x=6b+k where we obtain a convenient representation of natural numbers: n=42b+7k+i=42b+θ.

It is not difficult to verify that all prime numbers (Pn) except for 2 and 3 belong to the following two AP's ratio r=6: x=0, 1, . . . : AP₁=6x+5, AP₂=6x+7. Evidently, these two AP's also contain compose numbers (Cn). This superset of natural numbers represents candidates of primes (Cp). Considering the representation of natural numbers in terms of b, k and i, we obtain Cp given by the superset of natural numbers such that i+k=5 or 7 or 11, n≥5. Since each family of natural numbers is crossed by two AP's, such as n=42b+7k+i=42b+θ, under the restrictions of i and k, we obtain two values of k and θ per family (i=1, 2, . . . , 6).

Cp can be organized in subdivisions. Restricting to natural numbers such as i≠0 and i+k=5 or 7 or 11, we obtain 6 linear equations named families of natural numbers n=7x+i, AP's ratio r=7, crossed by 2 AP's ratio r=6. As a result, there are 12 groups of Cp n=42b+7k+i=42b+θ, which is equivalent to 6 families by transformation x=6b+k. Cp families are the basis for the construction of common denominators for SFq.

Constructed series of common denominators for SFq can be tested and validated in warm-blooded mammalian subjects and patients by the exposure of single, a series or combination of highly specific frequency RF carrier signals predetermined by the new mathematical model described above as an important aspect of the invention. Validation results named representative Hdp variation values and the correlated SFq obtained by the integrated solution of the invention provides a permanent refinement and adjustment of the linear models, based on artificial intelligence methods employed for pattern recognition described above. The methodology describe above provides the identification and generation of infinite series of SFq correlated with a heath condition of a warm-blooded mammalian subject.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., +10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the claims include equivalents to the recited values, e.g., variations in the numerical quantity of such values that can occur but would be recognized to be equivalents by a person skilled in the art.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

1-18. (canceled)

19. A system for diagnosing a cancer in a patient, comprising:
a hemodynamic parameter (Hdp) monitoring system configured to detect, measure, and record a plurality of first values for each of a plurality of hemodynamic parameters exhibited by a patient during a non-exposure period and a plurality of second values for each of the plurality of hemodynamic parameters exhibited by the patient during or after an exposure period, wherein the exposure period comprises a time period in which the patient is exposed to one or more electromagnetic signals having amplitude modulated frequencies (SFq), wherein the SFq are selected from a range of 0.01 Hz to 150 kHz;
an electrically powered frequency generator adapted to generate the one or more electromagnetic signals during the exposure period;
a probe coupled to the frequency generator, the probe configured to intrabuccally administer the one or more electromagnetic signals to the patient; and
a processing system coupled to the Hdp monitoring system and the frequency generator, the processing system configured to:
load the SFq from a library into the frequency generator to cause the probe to administer the one or more electromagnetic signals to the patient during the exposure period,
identify a hemodynamic response pattern for each of the plurality of hemodynamic parameters in response to exposure to the one or more electromagnetic signals based on the plurality of first values to the plurality of second values, and
diagnose the form of cancer in the patient by identifying the cancer that corresponds to the identified hemodynamic response pattern.

20. The system of claim 19, wherein the plurality of hemodynamic parameters comprise one or more of RR interval, heart rate, systolic blood pressure, diastolic blood pressure, median blood pressure, pulse pressure, stroke volume, cardiac output, and total peripheral resistance.

21. The system of claim 19, wherein the processing system is further configured to actuate the frequency generator to generate one or more highly specific frequency radio frequency (RF) carrier signals based on at least the plurality of first values for each of the plurality of hemodynamic parameters.

22. The system of claim 19, wherein the frequency generator comprises a programmable generator.

23. The system of claim 22, wherein the programmable generator comprises one or more controllable generator circuits, wherein each controllable generator circuit is configured to generate one or more highly specific frequency RF carrier signals.

24. The system of claim 23, wherein each controllable generator circuit comprises an amplitude modulation (AM) frequency control signal generator configured to control amplitude modulated variations of the one or more highly specific frequency RF carrier signals.

25. The system of claim 22, further comprising:
a storage medium adapted to store one or more electromagnetic field amplitude modulated frequencies (SFq),
wherein the processing system is further configured to retrieve one or more SFq from the storage medium based on the plurality of first values for each of the plurality of hemodynamic parameters and actuate the programmable generator to generate highly specific frequency RF carrier signals based on the one or more SFq.

26. A system for establishing hemodynamic parameter marker values for comparison with patient hemodynamic parameter values stored during treatment of a patient, comprising:
a hemodynamic parameter (Hdp) monitoring system configured to detect, measure and store a plurality of first values for a plurality of hemodynamic parameters exhibited by one or more subjects during a basal or non-exposure period and a plurality of second values for the plurality of hemodynamic parameters exhibited by the one or more subjects during or after an exposure period in which the one or more subjects are exposed to low-energy electromagnetic carrier output signals having amplitude modulated frequencies (SFq), wherein the SFq are selected from a range of 0.01 Hz to 150 kHz;
an electrically powered frequency generator adapted to be actuated to generate the low-energy electromagnetic carrier output signals for exposing or applying the low-energy electromagnetic carrier output signals to the one or more subjects during the exposure period;
a probe coupled to the frequency generator, the probe configured to intrabuccally administer the one or more electromagnetic signals to the one or more subjects, wherein the one or more subjects have a form of cancer; and
a processing system coupled to the Hdp monitoring system and the frequency generator, the processing system configured to:
load the SFq from a library into the frequency generator to cause the probe to administer the one or more electromagnetic signals to the patient during the exposure period,
identify representative Hdp variation values for the plurality of hemodynamic parameters in response to exposure to the one or more electromagnetic signals based on the plurality of first values to the plurality of second values, and
store the representative Hdp variation values in association with the form of cancer.

27. The system of claim 26, wherein the plurality of hemodynamic parameters include one or more of RR interval, heart rate, systolic blood pressure, diastolic blood pressure, median blood pressure, pulse pressure, stroke volume, cardiac output, and total peripheral resistance.

28. The system of claim 26, wherein the frequency generator comprises a programmable generator.

29. The system of claim 28, wherein the programmable generator comprises one or more controllable generator circuits, wherein each controllable generator circuit is configured to generate one or more highly specific frequency RF carrier signals, optionally wherein each controllable generator circuit comprises an amplitude modulation (AM) frequency control signal generator configured to control amplitude modulated variations of the one or more highly specific frequency RF carrier signals.

30. The system of claim 28, further comprising:
a storage medium adapted to store one or more electromagnetic field amplitude modulated frequencies (SFq),
wherein the processing system is further configured to retrieve one or more SFq from the storage medium based on the plurality of first values for each of the plurality of hemodynamic parameters and actuate the programmable generator to generate highly specific frequency RF carrier signals based on the one or more SFq.
